(19) United States
(12) Patent Application Publication
(10) Pub. No.: US 2022/0152114 A1
(43) Pub. Date: May 19, 2022

(54) AUTOLOGOUS THYMIC TISSUE TRANSPLANTATION

(71) Applicant: UNIVERSITY OF PITTSBURGH - OF THE COMMONWEALTH SYSTEM OF HIGHER EDUCATION, Pittsburgh, PA (US)

(72) Inventor: Eric LAGASSE, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 17/665,037

(22) Filed: Feb. 4, 2022

Related U.S. Application Data
(63) Continuation of application No. PCT/US2020/044940, filed on Aug. 5, 2020.
(60) Provisional application No. 62/882,887, filed on Aug. 5, 2019.

(51) Int. Cl. A61K 35/26; A61P 37/00

(52) U.S. Cl. CPC A61K 35/26; A61P 37/00

(57) ABSTRACT

The present disclosure provides methods and kits for preserving or restoring thymic functions of a subject in need thereof. The methods and kits disclosed herein include delivering an autologous thymic tissue into at least one lymph node of the subject.

### 1. CROSS-REFERENCE TO RELATED APPLICATION

This application is a Continuation of International Patent Application No. PCT/US2020/044940 filed Aug. 5, 2020, which claims priority to U.S. Provisional Application Ser. No. 62/882,887 filed Aug. 5, 2019, the contents of each of which are hereby incorporated by reference in its entirety.

### 2. TECHNICAL FIELD

The present disclosure provides methods for preserving or restoring thymic functions of a subject (e.g., a human subject) in need thereof, for example, after thymectomy surgery.

### 3. BACKGROUND

The thymus is a lymphoid organ of the immune system, which can produce and mature T cells. A thymectomy procedure is often performed to treat various thymus-related disorders, where the thymus is surgically removed. Thymectomy can be an effective treatment option for patients who have myasthenia gravis, thymoma, or a tumor of the thymus gland.

As the thymus is known to be most active during fetal and early postnatal life, its size and activity can start decreasing after the first years of life. Thymectomy can be routinely performed in certain children (e.g., neonates and infants) for congenital heart defect correction. As the thymus gland can be located under the breastbone blocking paths to the heart, partial or total thymectomy can be performed to have clear access and visualization of the heart and great vessels. In the United States, about 20,000 infants receive thymectomy each year.

However, thymectomy is believed to eliminate the main source for T-cell maturation and can cause serious long-term clinical impact, such as autoimmune diseases, cancer, infections, atopic diseases, and premature immunologic aging. Despite the popularity of thymectomy, no sufficiently effective treatment is available for children who have had thymectomy.

Thus, there remains a need for effective treatments for preserving or restoring the thymic functions of a subject who has had a thymectomy. The presently disclosed subject provides such treatments.

### 4. SUMMARY OF THE INVENTION

The present disclosure provides techniques and kits for preserving or restoring thymic functions of a subject (e.g., a human subject) in need thereof. The presently disclosed subject matter is based, at least in part, on the discovery that children, especially neonates and infants, that have had a thymectomy can develop serious long-term clinical disorders and diseases including autoimmune diseases, cancer, infections, atopic diseases, and premature immunologic aging.

In one aspect, the present disclosure provides a method for preserving or restoring thymic functions of a subject that has received or is receiving a thymectomy surgery. The method includes delivering thymic tissue into at least one lymph node of the subject, where the thymic tissue is autologous to the subject.

In certain embodiments, the subject has a congenital heart defect. In certain embodiments, the subject has received or is receiving an open heart surgery.

In certain embodiments, the subject has received or is receiving a thymectomy surgery. In non-limiting embodiments, the thymectomy surgery was performed when the subject was a neonate or an infant.

In certain embodiments, the subject is a human subject. In certain embodiments, the subject is a neonate or an infant. In certain embodiments, the subject is a child, an adult, or an adolescent.

In certain embodiments, the thymic tissue is obtained from the subject during the thymectomy surgery. In certain embodiments, the thymic tissue is obtained from the subject before the thymectomy surgery.

In certain embodiments, the thymic tissue is minced thymic fragments. In certain embodiments, the thymic tissue is cultured ex vivo before the delivery. In certain embodiments, the thymic tissue is cultured ex vivo for at least 24 hours before the delivery.

In certain embodiments, the thymic tissue is delivered into the lymph node of the subject through a needle.

In certain embodiments, the thymic tissue is delivered into the lymph node of the subject through a needle.

In certain embodiments, the thymic tissue is delivered into the lymph node during the thymectomy surgery. In certain embodiments, the thymic tissue is delivered into the lymph node after the thymectomy surgery.

In certain embodiments, the thymic tissue is cryopreserved before delivered into the lymph node. In certain embodiments, the cryopreserved thymic tissue is delivered into the lymph node after the thymectomy surgery. In certain embodiments, the cryopreserved thymic tissue is delivered into the lymph node about 1 week, about 2 weeks, about 3 weeks, about 4 weeks, about 2 months, about 4 months, about 6 months, about 1 year, about 2 years, about 3 years, about 4 years, about 5 years, about 10 years, about 15 years, about 20 years, or more, after the thymectomy surgery.

In certain embodiments, the thymic tissue is in an amount effective to restore the thymic function of the subject. In certain embodiments, the thymic tissue is in an amount effective to expand in the lymph node, where the expanded thymic tissue restores the thymic function of the subject.

In certain embodiments, the thymic tissue is in an amount of at least about 0.1 gram. In certain embodiments, the thymic tissue is in an amount of up to about 20 grams. In certain embodiments, the thymic tissue is in a size of at least about 0.1 cm³. In certain embodiments, the thymic tissue is in a size of up to about 20 cm³.

In certain embodiments, the thymic tissue is delivered into at least one, at least two, at least three, at least four, at least five, at least six, at least seven, at least eight, at least nine, or at least ten lymph nodes of the subject. In certain embodiments, the thymic tissue is a cryopreserved thymic tissue. In non-limiting embodiments, at least about a third of the full thymus weight of the subject is delivered into the at least one lymph node of the subject.

In another aspect, the present disclosure provides a kit for preserving or restoring thymic functions of a subject. The kit includes thymic tissue and tools for delivery of the thymic tissue to at least one lymph node of the subject, where the thymic tissue is autologous to the subject.

In certain embodiments, the kit further includes a solution, where the thymic tissue is provided in the solution. In certain embodiments, the solution includes a pharmaceutically acceptable excipient, a pharmaceutically acceptable diluent, or a pharmaceutically acceptable carrier.

In certain embodiments, the thymic tissue is in an amount effective to restore the thymic function of the subject. In certain embodiments, the thymic tissue is in an amount effective to expand in the lymph node, wherein the expanded thymic tissue restores the thymic function of the subject.

In certain embodiments, the kit further includes instructions that include delivery of the thymic tissue to the at least one lymph node of the subject. In non-limiting embodiments, the instructions include delivery of least about a third of the full thymus weight of the subject into the at least one lymph node of the subject.

In certain embodiments, the tools for delivery of the thymic tissue include needles and tools that are required for minimally invasive procedures.

In certain embodiments, the subject has received or is receiving a thymectomy surgery.

### 5. BRIEF DESCRIPTION OF THE DRAWINGS

FIGS. 1A-1C provide a description of wild-type newborn thymi collected and transplanted. FIG. 1A shows a bar graph of the weight of BALB/c wild type newborn thymi used for transplantation in accordance with the present disclosure. FIG. 1B shows a bar graph of the number of BALB/c nude mice transplanted with ¼, ½, or 1 lobe of the thymus, or the whole thymus in accordance with the present disclosure. FIG. 1C is a bar graph showing how many BALB/c nude mice were transplanted with each dose of thymus expressed in mg in accordance with the present disclosure.

FIGS. 2A-2E illustrate flow cytometric analyses of peripheral blood T cells four months after transplantation. FIG. 2A shows flow cytometry dot plots of CD3+ T cell percentages in an untransplanted (UnTx) BALB/c nude mouse, thymus-transplanted (Tx) BALB/c nude mice, and a BALB/c wild type mouse in accordance with the present disclosure. Cells are first gated for singlets, followed by live cells, CD45+ cells, and finally, for CD3 expression. FIG. 2B shows scatter dot plots with means of the percentage of CD45+ cells that are CD3+ in accordance with the present disclosure. FIG. 2C shows scatter dot plots with means of the percentage of CD3+ cells that are CD4+ in accordance with the present disclosure. FIG. 2D shows scatter dot plots with means of the percentage of CD3+ cells that are CD8+ in accordance with the present disclosure. FIG. 2E shows scatter dot plots with means and SEM of the percentage of CD45+ cells that are CD3+ T cells in thymus-transplanted BALB/c nude mice in accordance with the present disclosure.

FIGS. 3A-3B show flow cytometric analyses of peripheral blood T cells five months after transplantation. FIG. 3A shows a bar graph with a mean and standard deviation of the percentage of CD45+ cells that are CD3+ T cells in ¼, ½, 1, or 2 thymic lobes-transplanted BALB/c nude mice in accordance with the present disclosure. FIG. 3B shows a bar graph of the percentages of T cells expressing TCR-Vβ variants in mice as in FIG. 3A. A BALB/c wild type mouse represents a positive control (CNTRL).

FIGS. 4A-4B show lymph node weights six months after transplantation of thymic tissue fragments. FIG. 4A shows scatter dot plots with mean of lymph node (LN) weights in thymic lobes transplanted BALB/c nude mice in accordance with the present disclosure. FIG. 4B shows scatter dot plots with mean and SEM of lymph node (LN) weights in thymus-transplanted BALB/c nude mice in accordance with the present disclosure.

FIGS. 5A-5H show flow cytometry dot plots showing gating approach using flow cytometry for Effector/Effector Memory T-Cells (CD4+CD3+ CD44+CD62L−) and (CD8+CD3+ CD44+CD62L−); Central Memory T-Cells (CD4+CD3+ CD44+CD62L+) and (CD8+CD3+ CD44+CD62L+); Activated Effector T-Cells (CD4+CD3+ CD44−CD62L−) and (CD8+CD3+ CD44−CD62L−); Naïve T-Cells (CD4+CD3+ CD44−CD62L+) and (CD8+CD3+ CD44−CD62L+) in accordance with the present disclosure. FIG. 5A illustrates a forward scatter vs side scatter gate. FIG. 5B illustrates a gate for single cells. FIG. 5C illustrates a gate for live cells. FIG. 5D illustrates a gate for CD45+ cells. FIG. 5E illustrates a gate for CD3+ cells. FIG. 5F illustrates gates for CD4+ and CD8+ cells. FIG. 5G illustrates gating of CD4+ cells for naïve, activated effector, effector memory, and central memory cells. FIG. 5H illustrates gating of CD8+ cells for naïve, activated effector, effector memory, and central memory cells.

FIGS. 6A-6E show flow cytometric analyses of peripheral blood T cell subsets 7 weeks after transplantation of thymic tissue fragments into lymph nodes of adult and aged mice. FIG. 6A shows flow cytometry dot plots of naïve, effector, and effector memory, and central memory CD4+ T cell subpopulations in adult or old C57BL/6J females with or without ¼ thymus lobe transplantation (Tx) in accordance with the present disclosure. FIGS. 6B-6E show scatter dot plots with mean and SEM of the percentages of CD4+ T cells that are activated CD4+ T cell effectors (FIG. 6B), CD4+ effector memory T cells (FIG. 6C), CD4+ central memory T cells (FIG. 6D) and CD4+ naive T cells (FIG. 6E) in adult or old C57BL/6J females and males with or without ¼ thymus lobe transplantation in accordance with the present disclosure.

### 6. DETAILED DESCRIPTION

Non-limiting embodiments of the present disclosure are described by the present specification and Examples. For purposes of clarity of disclosure and not by way of limitation, the detailed description is divided into the following subsections:

6.1 Definitions;

6.2 Methods of treatment; and

6.3 Kits.

### 6.1 Definitions

The terms used in this specification generally have their ordinary meanings in the art, within the context of this disclosed subject matter and in the specific context where each term is used. Certain terms are discussed below, or elsewhere in the specification, to provide additional guidance to the practitioner in describing the compositions and methods of the disclosed subject matter and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing,” and “comprising” are interchangeable, and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

An adult human subject is a subject that has attained an age of at least about 18 years. An adult non-human subject is a subject that has attained sexual maturity. In certain embodiments, a neonate is a human subject that has attained an age of at most about 1 month. In certain embodiments, an infant is a human subject that has attained an age of between about 1 month and about 2 years. In certain embodiments, a child is a human subject that has attained an age of between about 2 years and about 12 years. In certain embodiments, an adolescent is a human subject that has attained an age of between about 12 years and about 18 years.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of the extent of disease, stabilized (i.e., not worsening) state of disease, prevention of disease, delay or slowing of disease progression, and/or amelioration or palliation of the disease state. The decrease can be an at least 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment. In certain embodiments, the subject matter disclosed herein are used to restore the thymus function in a subject (e.g., a human subject, a neonate, an infant, a child, an adolescent, or an adult). In certain embodiments, the subject matter disclosed herein are used to increase the level of circulating T cells in a subject (e.g., naïve T cells in peripheral blood).

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the term “pharmaceutically acceptable” can refer to those compounds, materials, compositions, and/or dosage forms which are, within the scope of sound medical judgment, suitable for use in contact with the tissues of the subject (e.g., human subject or non-human animals) without excessive toxicity, irritation, allergic response, or other problem or complication, commensurate with a reasonable benefit/risk ratio.

As used herein, the term “pharmaceutically-acceptable excipient” can refer to a pharmaceutically-acceptable material, composition or vehicle, such as a liquid or solid filler, diluent, carrier, manufacturing aid (e.g., lubricant, talc magnesium, calcium or zinc stearate, or stearic acid), or solvent encapsulating material, involved in carrying or transporting the subject compound, materials, or cells, to an organ or portion of the body. Each excipient must be “acceptable” in the sense of being compatible with the other ingredients of the formulation, e.g., the cells to be transplanted, and not injurious to the subject.

### 6.2 Methods of Treatment

The present disclosure provides methods for preserving or restoring thymic functions of a subject in need thereof. The methods include delivering a thymic tissue (e.g., an autologous thymic tissue) into at least one lymph node of the subject for preserving, increasing, or restoring thymic functions of the subject. In certain embodiments, the methods disclosed herein can prevent or reduce short or long-term clinical impacts of thymectomy surgery, such as autoimmune disease, cancer, infection, atopic disease, premature immunologic aging, or a combination thereof.

### 6.2.1 Thymic Tissues

In certain embodiments, the subject has received or is receiving thymectomy surgery. In certain embodiments, the autologous thymic tissue delivered into the subject is obtained from the subject during thymectomy surgery. In certain embodiments, the autologous thymic tissue delivered into the subject is obtained from the subject before the thymectomy surgery. In certain embodiments, the thymectomy surgery was performed when the subject is a neonate, an infant, or a child.

In some embodiments, thymic tissue delivered to a subject is not autologous, e.g., is allogeneic. In some embodiments, thymic tissue is from a donor that is matched to the subject at one or more human leukocyte antigen (HLA) alleles (e.g., matched at one or both copies of 1, 2, 3, 4, 5, or 6 HLA alleles). A thymic tissue can be from a donor that is partially HLA matched, fully HLA matched, or haploidentical to the subject. A subject can be administered an immunosuppressive regimen to reduce the likelihood of transplant rejection. Non-limiting examples of drugs that can be administered as part of an immunosuppressive regimen include mTOR inhibitors (e.g., rapamycin), inhibitors of CD28-B7 (e.g., CTLA4-Ig), inhibitors of CD40-CD40L (e.g., MR1), steroids (such as corticosteroids, dexamethasone, and prednisone), Cox-1 and Cox-2 inhibitors, macrolide antibiotics (such as rapamycin and tacrolimus), cyclosporine, azathioprine, Atgam, Thymoglobulin, OKT3, Basiliximab, Solumedrol, Daclizumab, Mycophenolate Mofetil, Prograf, and other substances that limit, reduce, or suppress B-cell, T-cell, and/or other innate immune activity. An immunosuppressive regimen can comprise one drug or any combination of suitable drugs administered so as to achieve the desired effect.

Thymic tissue for delivery can be prepared using any suitable techniques known in the art including, for example, but not limited to, techniques described in Market et al., Blood 102, 1121-1130 (2003), the contents of which are incorporated by reference herein in its entirety. In certain embodiments, the thymic tissue harvested from a subject is processed (e.g., minced or sliced) into small fragments and resuspended in a liquid to form a solution to be delivered into a lymph node. In some embodiments, grafts comprising thymic fragments are superior to single cell suspension thymic grafts for generating a functional ectopic thymus. For example, a graft comprising thymic fragments can result in a greater amount of engrafted thymic tissue, an increased proportion or number of circulating T cells, an increased proportion or number of circulating naïve T cells, or advantages in other parameters disclosed herein. The solution can be preferably sterile. The solution can be stable under the conditions of manufacture and storage, and can be preserved against contamination of microorganisms such as bacteria and fungi through the use of, for example, parabens, chlorobutanol, phenol, ascorbic acid, thimerosal, and the like.

In certain embodiments, the solution includes different types of cells that constitute the thymic tissue. In certain embodiments, the solution further includes pharmaceutically acceptable excipients, diluents, or carriers. Pharmaceutically acceptable carriers and diluents include saline, aqueous buffer solutions, solvents, and/or dispersion media. Non-limiting examples of pharmaceutically acceptable excipients that can be used with the presently disclosed methods include: sugars, such as lactose, glucose and sucrose; starches, such as corn starch and potato starch; cellulose, and its derivatives, such as sodium carboxymethyl cellulose, methylcellulose, ethyl cellulose, microcrystalline cellulose and cellulose acetate; powdered tragacanth; malt; gelatin; lubricating agents, such as magnesium stearate, sodium lauryl sulfate and talc; cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol (PEG); esters, such as ethyl oleate and ethyl laurate; agar; buffering agents, such as magnesium hydroxide and aluminum hydroxide; alginic acid; pyrogen free water; isotonic saline; Ringer's solution; ethyl alcohol; pH buffered solutions; polyesters, polycarbonates and/or polyanhydrides; bulking agents, such as polypeptides and amino acids; serum component, such as serum albumin, HDL and LDL; C2-C12 alcohols, such as ethanol; and other non-toxic compatible substances employed in pharmaceutical formulations.

In certain embodiments, the thymic tissue is cultured ex vivo before being processed or delivered into the subject. Any culturing media known in the art for culturing organs can be used with the presently disclosed methods. In certain embodiments, the medium includes nutrients for sustaining the viability of the tissue, at least one antibiotic for preventing microbial infection of the thymic tissue, and a buffer system for maintaining an appropriate pH range of the medium. In certain embodiments, the medium includes F12 nutrient mixture 25 mM HEPES, 2 mM L-glutamine, 10% fetal bovine serum, 100 ug/mL streptomycin sulfate, 1 ug/mL gentamycin, and 100 ug/dL Amphotericin B. In certain embodiments, the thymic tissue is cultured in the medium in a 5% CO₂ incubator at 37° C. In certain embodiments, the thymic tissue is cultured ex vivo for at least about 2 hours, about 4 hours, about 12 hours, about 24 hours, about 2 days, about 3 days, about 4 days, about 5 days, about 6 days, about 7 days, about 8 days, about 10 days, about 2 weeks, about 3 weeks, about 4 weeks, about 5 weeks, about 6 weeks, about 7 weeks, about 8 weeks, about 9 weeks, or about 10 weeks, or more, before the thymic tissue is processed or delivered into the subject. In certain embodiments, the thymic tissue is cultured before cryopreservation, after cryopreservation, or a combination thereof. In certain embodiments, the thymic tissue is not cultured ex vivo before being processed and/or delivered into the subject.

In certain embodiments, the thymic tissue is cryopreserved before being processed or delivered into the subject. Any methods known in the art for cryopreserving thymic tissues can be used with the methods disclosed herein, for example, but not limited to, the methods disclosed in Jang et al., Integr Med Res. 2017 Mar; 6(1): 12-18, the contents of which are incorporated herein by reference in its entirety. In certain embodiments, the thymic tissue is cryopreserved for at least about 12 hours, about 24 hours, about 2 days, about 3 days, about 1 week, about 2 weeks, about 3 weeks, about 4 weeks, about 2 months, about 3 months, about 4 months, about 5 months, about 6 months, about 8 months, about 10 months, about 1 year, about 2 years, about 3 years, about 4 years, about 5 years, about 6 years, about 7 years, about 8 years, about 9 years, about 10 years, about 15 years, about 20 years, or more before delivered into the lymph node. In certain embodiments, the cryopreserved thymic tissue is delivered into the lymph node long after the thymectomy surgery. In certain embodiments, the thymic tissue is not cryopreserved before being processed and/or delivered into the subject.

In certain embodiments, the thymic tissue is delivered into the subject during the thymectomy surgery. In certain embodiments, the thymic tissue is delivered into the subject after the thymectomy surgery. In certain embodiments, the thymic tissue is delivered into the subject about 10 minutes, about 30 minutes, about 1 hour, about 2 hours, about 3 hours, about 6 hours, about 12 hours, about 24 hours, about 2 days, about 3 days, about 1 week, about 2 weeks, about 3 weeks, about 4 weeks, about 2 months, about 3 months, about 4 months, about 5 months, about 6 months, about 8 months, about 10 months, about 1 year, about 2 years, about 3 years, about 4 years, about 5 years, about 6 years, about 7 years, about 8 years, about 9 years, about 10 years, about 15 years, about 20 years, or more after the thymectomy surgery. In non-limiting embodiments, the thymic tissue is delivered into the subject while the thymectomy surgery is performed.

In certain embodiments, the thymic tissue delivered into the subject is in an amount effective to restore the thymic function of the subject. In certain embodiments, the thymic tissue delivered into the subject is in an amount effective to increase the thymic function of the subject. In certain embodiments, the thymic tissue delivered into the subject is in an amount effective to expand in the lymph node, e.g., where the expanded thymic tissue restores the thymic function of the subject. In certain embodiments, the thymic tissue delivered into the subject is in an amount effective to engraft into the lymph node. In certain embodiments, the thymic tissue delivered into the subject is in an amount effective to form an ectopic thymus tissue in the lymph node. In certain embodiments, the thymic tissue delivered into the subject is in an amount effective to increase the level of circulating T cells in the subject (e.g., T cells and/or naïve T cells in peripheral blood). In certain embodiments, the thymic tissue delivered into the subject is in an amount effective to increase the diversity of circulating T cells in the subject (e.g., increase the repertoire of TCRs and recognized cognate antigens). The effective amount can vary with the subject's history, age, condition, sex, as well as the severity and type of the medical condition in the subject, and administration of other pharmaceutically active agents.

In certain embodiments, the methods and systems are used to deliver thymic cells or thymus fragments into a lymph node of the subject, allowing the thymic cells or fragments to engraft and produce an ectopic thymus in the lymph node. In certain embodiments, the ectopic thymus restores the thymic function of the subject, e.g., supplements or augments one or more functions that a normal healthy thymus organ can perform. For example, but not by way of limitation, the ectopic thymus can participate in immunomodulation of the body for its participation in T cell growth, development, maturation, and selection. Production of the ectopic thymus in accordance with the present disclosure can find use in augmenting or modulating immune system function in subjects having immune system dysfunction, for instance, in subjects having received thymectomy surgery, e.g., partial or complete removal of thymus.

In certain embodiments, the present disclosure relates to the transplantation of thymic tissue from a neonatal or infant subject into a lymph node of the subject himself/herself. In some embodiments, the subject receives the transplantation at the time, or shortly after (e.g., within 24 hours, within one week, one month, or one year) the thymic tissue is obtained from the body. In some of these embodiments, the subject is a neonate or infant when receiving the transplantation into the lymph node. In some embodiments, the subject receives the transplantation years after the thymic tissue is taken from the subject. For instance, the subject is an adult, child, or adolescent when receiving the transplantation while the thymic tissue was taken from the subject when the subject was a neonate or infant.

In certain embodiments, the methods and systems that comprise transplantation of autologous thymic tissue from a neonate or infant into lymph node of a subject when the subject is a neonate, infant, or child are advantageous as compared to transplantation of embryonic thymic tissue or adult thymic tissue into an adult subject. Without wishing to be bound by a certain theory, the present methods and systems are advantageous because, among other advantages, transplantation of autologous neonatal thymic tissue into the lymph node of the subject at a young age (e.g., neonate, infant, or child) can provide an environment for the transplanted thymic cells to grow and function in that is similar to the environment in which a native neonatal thymus would grow and develop. Newborn mammals, e.g., humans, can have limited peripheral immune systems immediately after birth, with few T lymphocytes present in the peripheral tissues. Native thymus can thus be needed to continue to grow in size, in order to populate the peripheral immune system with lymphocytes (e.g., Naïve T lymphocytes). During this early neonatal development process, native thymus can experience dramatic changes at molecular and cellular levels due to its own development as well as the various influences from the subject, which is maturing at the same time, including the developing hormonal and immune systems of the subject. Unique molecular and cellular features of the neonatal thymic tissue have been reported. For example, gamma/delta T cells, which arise during fetal/neonatal thymic development, can exhibit no development or reduced development in the adult thymus (Ito et al., Proc. Natl. Acad. Sci. USA, (1989) 86:631). In addition, distinct populations of regulatory T cells (Tregs) are produced during the perinatal age-window in the thymus. These particular Tregs can play an essential role in maintaining self-tolerance and can be distinguishable from adult-derived Tregs based on molecular and activation profiles (Yang et al., Science. 2015 May 1; 348(6234): 589-594). For another example, CD4+ T cells derived from neonatal thymus can have functional properties that are distinct from adult-thymic derived cells (Becky Adkins, The Journal of Immunology, 2003, 171: 5157-5164). Such unique developmental environments and changes of the neonatal thymus at the early childhood period may not be present at other developmental stages, and yet can be important for the structural and functional maturation of thymus. As a result, the present methods and systems can provide a developmental environment that may not be present at other stages of the life of the subject, for example, when the subject is an adult. The methods and systems disclosed herein can be advantageous because, among other advantages, neonatal thymic tissue can be more developed as compared to embryonic thymic tissue, and also have better growth capability as compared to adult thymic tissue, which may have experienced age-related involution. Thus, the neonatal thymic tissue can have a better chance to grow into an ectopic thymic tissue that has sufficient mass and function to restore the development and maturation of the immune system (e.g., naïve T lymphocytes) in the subject.

In certain embodiments, expansion of thymic tissue includes engraftment, proliferation, differentiation, and/or growth of the thymic tissue in the lymph node to produce an ectopic thymic tissue in the lymph node. The produced ectopic thymic tissue is biologically active and has thymic functions. In certain embodiments, the thymic tissue expands in the lymph node, such that the mass of the ectopic tissue eventually produced is higher than the original mass of the thymic tissue delivered to the lymph node. In certain embodiments, the mass of the ectopic tissue eventually produced is at least about 1.1 times, 1.2 times, at least about 1.3 times, at least about 1.4 times, at least about 1.5 times, at least about 2 times, at least about 3 times, at least about 4 times, at least about 5 times, at least about 10 times, at least about 15 times, at least about 20 times, at least about 30 times, at least about 40 times, at least about 50 times, at least about 100 times, at least about 150 times, at least about 200 times, at least about 500 times, or at least about 1000 times of the original mass of the thymic tissue delivered to the lymph node.

In certain embodiments, vascularization can take place in the lymph node receiving the thymic tissue, e.g., there can be blood vessels infiltrating and forming vasculature network within the lymph node, and within the ectopic thymic tissue. In certain embodiments, the infiltrating vasculature network can form the basis of blood supply to the ectopic thymic tissue. In certain embodiments, the infiltrating vasculature network can help transport T cells to and from the ectopic thymic tissue. In certain embodiments, the lymphatic circulation system also serves as a transportation channel for the substances (e.g., T cells) produced by the ectopic tissue. The lymphatic system, in certain embodiments, can thus provide such substances to the blood circulation system via their crosstalk with other parts of the body.

In certain embodiments, the thymic tissue delivered into the subject is in an amount (e.g., an effective amount as disclosed herein) of at least about 0.1 gram, at least about 0.2gram, at least about 0.3 gram, at least about 0.4 gram, at least about 0.5 gram, at least about 0.6 gram, at least about 0.7 gram, at least about 0.8 gram, at least about 0.9 gram, at least about 1 gram, at least about 1.5 grams, at least about 2 grams, at least about 2.5 grams, at least about 3 grams, at least about 4 grams, at least about 5 grams, at least about 6 grams, at least about 7 grams, at least about 8 grams, at least about 9 grams, or at least about 10 grams. In certain embodiments, the thymic tissue delivered into the subject is in an amount (e.g., an effective amount as disclosed herein) of up to about 5 grams, up to about 10 grams, up to about 15 grams, or up to about 20 grams. In certain embodiments, the thymic tissue delivered into the subject is in an amount (e.g., an effective amount as disclosed herein) of about 0.1 gram, about 0.2 gram, about 0.3 gram, about 0.4 gram, about 0.5 gram, about 0.6 gram, about 0.7 gram, about 0.8 gram, about 0.9 gram, about 1 gram, about 2 grams, about 3 grams, about 4 grams, about 5 grams, about 6 grams, about 8 grams, about 10 grams, about 13 grams, about 15 grams, about 18 grams, about 20 grams, or more. Weight can be measured before processing, for example, before mincing thymic tissue and adding liquid go generate a suspension. In some embodiments, weight is measured after processing, for example, after mincing thymic tissue and adding liquid go generate a suspension. The amount of thymic tissue can be delivered to one lymph node or can be split between two or more lymph nodes as disclosed herein. The amount of thymic tissue can be administered in one dose or in two or more doses separated by any suitable period of time as disclosed herein. When two or more doses, the amount can be the total split between two or more doses, or the amount per dose.

In some embodiments, the thymic tissue is delivered into the subject in an amount (e.g., an effective amount as disclosed herein) that is relative to the body weight of the subject. For example, the thymic tissue can be delivered to the subject in an amount of at least about 0.001 mg/kg, at least about 0.005 mg/kg, at least about 0.01 mg/kg, at least about 0.05 mg/kg, at least about 0.1 mg/kg, at least about 0.5 mg/kg, at least about 1 mg/kg, at least about 5 mg/kg, at least about 10 mg/kg, at least about 15 mg/kg, at least about 20 mg/kg, at least about 25 mg/kg, at least about 30 mg/kg, at least about 40 mg/kg, at least about 50 mg/kg, at least about 60 mg/kg, at least about 70 mg/kg, at least about 80 mg/kg, at least about 90 mg/kg, at least about 100 mg/kg, at least about 110 mg/kg, at least about 120 mg/kg, at least about 130 mg/kg, at least about 140 mg/kg, at least about 150 mg/kg, at least about 160 mg/kg, at least about 170 mg/kg, at least about 180 mg/kg, at least about 190 mg/kg, at least about 200 mg/kg, at least about 250 mg/kg, at least about 500 mg/kg, at least about 1000 mg/kg, or at least about 1500 mg/kg. In some embodiments, the thymic tissue is delivered into the subject in an amount that is at most about 1 mg/kg, at most about 10 mg/kg, at most about 20 mg/kg, at most about 50 mg/kg, at most about 100 mg/kg, at most about 110 mg/kg, at most about 120 mg/kg, at most about 130 mg/kg, at most about 140 mg/kg, at most about 150 mg/kg, at most about 160 mg/kg, at most about 170 mg/kg, at most about 180 mg/kg, at most about 190 mg/kg, at most about 200 mg/kg, at most about 250 mg/kg, at most about 500 mg/kg, at most about 1000 mg/kg, or at most about 1500 mg/kg. In some embodiments, the thymic tissue is delivered into the subject in an amount that is about 0.001 mg/kg, about 0.005 mg/kg, about 0.01 mg/kg, about 0.05 mg/kg, about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 250 mg/kg, about 500 mg/kg, about 1000 mg/kg, or about 1500 mg/kg. An effective amount as disclosed herein can be an amount relative to the body weight of the subject. The amount of thymic tissue can be measured before processing, for example, before mincing thymic tissue and adding liquid go generate a suspension. The amount of thymic tissue can be delivered to one lymph node or can be split between two or more lymph nodes as disclosed herein. The thymic tissue of the given size can be administered in one dose or in two or more doses separated by any suitable period of time as disclosed herein. When two or more doses, the amount of thymic tissue can be the total split between two or more doses, or the amount per dose.

In certain embodiments, the thymic tissue delivered into the subject is in a size of at least about 0.1 cm³, at least about 0.2 cm³, at least about 0.3 cm³, at least about 0.4 cm³, at least about 0.5 cm³, at least about 0.6 cm³, at least about 0.7 cm³, at least about 0.8 cm³, at least about 0.9 cm³, at least about 1 cm³, at least about 1.5 cm³, at least about 2 cm³, at least about 2.5 cm³, or at least about 3 cm³. In certain embodiments, the thymic tissue delivered into the subject is in a size of up to about 5 cm³, up to about 10 cm³, up to about 15 cm³, or up to about 20 cm³. In certain embodiments, the thymic tissue delivered into the subject is in a size of about 0.1 cm³, about 0.2 cm³, about 0.3 cm³, about 0.4 cm³, about 0.5 cm³, about 0.6 cm³, about 0.7 cm³, about 0.8 cm³, about 0.9 cm³, about 1 cm³, about 2 cm³, about 3 cm³, about 4 cm³, about 5 cm³, about 6 cm³, about 8 cm³, about 10 cm³, about 13 cm³, about 15 cm³, about 18 cm³, about 20 cm³, or more. An effective amount as disclosed herein can be a expressed as a size. Size can be measured before processing, for example, before mincing thymic tissue and adding liquid go generate a suspension. In some embodiments, size is measured after processing, for example, after mincing thymic tissue and adding liquid go generate a suspension. The thymic tissue of the given size can be delivered to one lymph node or can be split between two or more lymph nodes as disclosed herein. The thymic tissue of the given size can be administered in one dose or in two or more doses separated by any suitable period of time as disclosed herein. When two or more doses, the thymic tissue of the given size can be the total split between two or more doses, or the amount per dose.

In certain embodiments, the thymic tissue delivered into the subject is in a liquid suspension. In some embodiments, the thymic tissue delivered into the subject in a liquid suspension in a volume of at least about 0.1 mL, at least about 0.2 mL, at least about 0.3 mL, at least about 0.4 mL, at least about 0.5 mL, at least about 0.6 mL, at least about 0.7 mL, at least about 0.8 mL, at least about 0.9 mL, at least about 1 mL, at least about 1.5 mL, at least about 2 mL, at least about 2.5 mL, at least about 3 mL, at least about 4 mL, at least about 5 mL, at least about 6 mL, at least about 7 mL, at least about 8 mL, at least about 9 mL, or at least about 10 mL (for example, per lymph node, or in total split between two or more lymph nodes). In certain embodiments, the thymic tissue delivered into the subject is in a volume of up to about 5 mL, up to about 10 mL, up to about 15 mL, or up to about 20 mL (for example, split between two or more lymph nodes). In certain embodiments, the thymic tissue delivered into the subject is in a volume of about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 2 mL, or about 3 mL per lymph node. In certain embodiments, the thymic tissue delivered into the subject is in a volume of about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 8 mL, about 10 mL, about 13 mL, about 15 mL, about 18 mL, about 20 mL, or more, split between two or more lymph nodes. The volume can be administered in one dose or in two or more doses separated by any suitable period of time as disclosed herein. When two or more doses, the volume can be the total volume split between two or more doses, or the volume per dose. An effective amount as disclosed herein can be expressed as a volume.

A subject can receive one dose of thymic tissue or two or more doses separated by any suitable period of time. Doses can be separated by a consistent interval or an irregular interval. Two doses can be separated by, for example, about half an hour, about 1 hour, about 2 hours, about 3 hours, about 4 hours, about 5 hours, about 6 hours, about 8 hours, about 10 hours, about 12 hours, about 16 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 16 weeks, about 4 months, about 5 months, about 6 months, about 7 months, about 8 months, about 9 months, about 10 months, about 11 months, about 12 months, about 14 months, about 16 months, about 18 months, about 20 months, about 22 months, about 24 months, about 30 months, about 36 months, about 4 years, about 5 years, about 6 years, about 7 years, about 8 years, about 9 years, or about 10 years. In some embodiments, a thymic tissue can be administered to a subject in 1, 2, 3, 4, 5, 6, 7, 8, 9, 10, 11, 12, 13, 14, 15, 16, 17, 18, 19, or 20 doses.

In certain embodiments, the disclosed subject matter can allow the subject to have an increased circulating blood T-cell count and restore normal immune functions. In non-limiting embodiments, at least about a third of the full thymus weight of the subject can be implanted to have a high circulating blood T-cell count and restore normal immune. For example, over about 3 mg of thymus tissue can be delivered into a mouse subject, which has an average of about 8.5 mg full-size newborn thymus. In the human subject, about 7 gram (gr), about 8 gr, about 9 gr, or about 10 gr of thymus tissue can be delivered into the human subject, who has an average 25 gr full-size pediatric thymus. A full thymus weight can be a full weight of a neonatal thymus. A full thymus weight can be a full weight of an infant thymus. A full thymus weight can be a full weight of a child thymus. A full thymus weight can be a full weight of a child thymus. A full thymus weight can be a full weight of an adult thymus. In some embodiments, a full thymus weight can be a full thymus weight for a normal (e.g., healthy or immunocompetent) individual that is a similar age as the subject. In some embodiments, a full thymus weight can be a full thymus weight for a normal individual that is a different age than the subject.

In certain embodiments, the disclosed subject matter can increase the blood circulating CD3+ T cells and restore the naïve T-cell population of a subject. For example, a subject can have a lymphopenia, which can cause a decrease in naïve T cells with a concomitant increase in the memory T-cell population. A quantitative defect in the T-cell compartment can also affect the naïve T-cell population. The disclosed subject matter can allow such subject to restore the naïve T-cell population by increasing the blood circulating CD3+ T cells. In some embodiments, the disclosed subject matter can reduce T cell senescence in the subject.

In some embodiments, the frequency of CD45+ peripheral blood cells that are T cells can be increased or maintained in a subject by compositions and methods disclosed herein. For example, in some cases the frequency of CD45+ peripheral blood cells that are T cells can be at least about 1%, at least about 5%, at least about 10%, at least about 15%, at least about 20%, at least about 30%, at least about 40%, at least about 50%, at least about 60%, or at least about 70% after delivering a thymic tissue to a lymph node as disclosed herein. In some embodiments, the frequency of CD45+ peripheral blood cells that are T cells can be increased by at least about 1%, at least about 2%, at least about 5%, at least about 10%, at least about 15%, at least about 20%, at least about 30%, at least about 40%, at least about 50%, at least about 60%, at least about 70%, or more, relative to before delivering a thymic tissue as disclosed herein, or relative to a comparable subject that does not receive a thymic tissue. For example, the frequency of CD45+ peripheral blood cells that are T cells can be 1% before delivering a thymic tissue as disclosed herein, and can be increased by 10% to achieve a frequency of 11% after a thymic tissue is delivered.

In some embodiments, the concentration of T cells in peripheral blood of a subject can be increased by compositions and methods disclosed herein. For example, in some cases the concentration of T cells in peripheral blood of a subject can be increased by at least about 5%, at least about 10%, at least about 15%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, at least about 10-fold, at least about 20-fold, at least about 50-fold, at least about 100-fold, at least about 200-fold, at least about 500-fold, at least about 1000-fold, or at least about 10,000-fold, or more , e.g., relative to before delivering a thymic tissue as disclosed herein, or relative to a comparable subject that does not receive a thymic tissue. The concentration can be expressed, for example, as the number of cells per cubic millimeter, microliter, deciliter, milliliter, or liter.

Methods of identifying T cells are known in the art. In some embodiments, T cells are identified based on being CD3+, CD3+ CD4+, CD3+ CD8+, CD3+ CD4+ CD45+, or CD3+ CD8+ CD45+.

In some embodiments, the frequency of CD3+ peripheral blood cells that are naïve T cells can be increased or maintained in a subject by methods disclosed herein. For example, in some cases the frequency of CD3+ peripheral blood cells that are naïve T cells can be at least about 1%, at least about 5%, at least about 10%, at least about 15%, at least about 20%, at least about 30%, at least about 40%, at least about 50%, at least about 60%, at least about 70%, or at least about 80% after delivering a thymic tissue to a lymph node as disclosed herein. In some embodiments, the frequency of CD3+ peripheral blood cells that are naïve T cells can be increased by at least about 1%, at least about 2%, at least about 5%, at least about 10%, at least about 15%, at least about 20%, at least about 30%, at least about 40%, at least about 50%, at least about 60%, at least about 70%, or more, relative to before delivering a thymic tissue as disclosed herein, or relative to a comparable subject that does not receive a thymic tissue. For example, the frequency of CD3+ peripheral blood cells that are naïve T cells can be 1% before delivering a thymic tissue as disclosed herein, and can be increased by 10% to achieve a frequency of 11% after a thymic tissue is delivered.

In some embodiments, the concentration naïve T cells in the peripheral blood of a subject can be increased by compositions and methods disclosed herein. For example, in some cases the concentration of naïve T cells in peripheral blood of a subject can be increased by at least about 5%, at least about 10%, at least about 15%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, at least about 10-fold, at least about 20-fold, at least about 50-fold, at least about 100-fold, at least about 200-fold, at least about 500-fold, at least about 1000-fold, or at least about 10,000-fold, or more, e.g., relative to before delivering a thymic tissue as disclosed herein, or relative to a comparable subject that does not receive a thymic tissue. The concentration can be expressed, for example, as the number of cells per cubic millimeter, microliter, deciliter, milliliter, or liter.

Methods of identifying naïve T cells, other subsets disclosed herein, and equivalent populations in other species (e.g., humans) are known in the art. In some embodiments, naïve T cells are CD3+ CD4+ CD44− CD62L+ or CD3+ CD8+ CD44− CD62L+. In some embodiments, naïve T cells are CD3+ CD4+ CD45RA+ CCR7+, CD3+ CD8+ CD45RA+ CCR7+, or any combination of CD3+, CD4+, CD8+, CD45RA+, CCR7+, CD45RO−, and CD27+.

In some embodiments, compositions and methods of the disclosure can be used to achieve a certain CD4:CD8 T cell ratio in a subject (for example, a CD4:CD8 ratio of circulating total T cells, naïve T cells, effector T cells, effector/memory T cells, central memory T cells, or a combination thereof). For example, a CD4:CD8 ratio achieved by compositions and methods disclosed herein can be at least about 1:1, at least about 1.1:1, at least about 1.2:1, at least about 1.3:1, at least about 1.4:1, at least about 1.5:1, at least about 1.6:1, at least about 1.7:1, at least about 1.8:1, at least about 1.9:1, or at least about 2:1. In some embodiments, a CD4:CD8 ratio achieved by compositions and methods disclosed herein can be at most about 2:1, at most about 2.1:1, at most about 2.2:1, at most about 2.3:1, at most about 2.4:1, at most about 2.5:1, at most about 2.6:1, at most about 2.7:1, at most about 2.8:1, at most about 2.9:1, at most about 3:1, at most about 2.5:1, at most about 3:1, at most about 4:1, or at most about 5:1. In some embodiments, a CD4:CD8 ratio achieved by compositions and methods disclosed herein is about 2:1.

The effects of compositions and methods of the disclosure on outcomes discloses herein can be evaluated any suitable amount of time after delivering a thymic tissue to a lymph node of a subject. For example, effects on the number and/or frequency of peripheral blood T cells, the concentration and/or frequency of naïve peripheral blood T cells, the CD4:CD8 ratio, lymph node weight, expansion of the thymic tissue in the lymph node, or any other outcome disclosed herein can be evaluated about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 16 weeks, about 4 months, about 5 months, about 6 months, about 7 months, about 8 months, about 9 months, about 10 months, about 11 months, about 12 months, about 14 months, about 16 months, about 18 months, about 20 months, about 22 months, about 24 months, about 30 months, about 36 months, about 4 years, about 5 years, about 6 years, about 7 years, about 8 years, about 9 years, or about 10 years after delivering a thymic tissue to a lymph node of a subject.

Any methods known the art for delivering agents, materials, or cells to internal organs of a subject can be used with the presently disclosed subject matter. In certain embodiments, the thymic tissue is delivered to the lymph node of the subject through a needle. In certain embodiments, the needle is a microinjection needle. In certain embodiments, the methods disclosed herein include introducing a needle into the lymph node of the subject. Minimally invasive procedures are performed under the guidance of ultrasound and can have the benefit of operating with less damage to the body than with open surgery. Additionally, minimally invasive procedures can be associated with less pain, a shorter hospital stay, and fewer complications than open surgeries. In certain embodiments, the presently disclosed methods are performed using a minimally invasive procedure. In certain embodiments, the minimally invasive procedure is performed under the guidance of ultrasound. Non-limiting examples of minimally invasive procedures that can be used include those disclosed in PCT Patent Application No. PCT/US2020/027783, which is incorporated herein by reference in its entirety.

### 6.2.2 Lymph Nodes

In certain embodiments, the thymic tissue is delivered into at least one lymph node of the subject. In certain embodiments, the thymic tissue is delivered into at least two, at least three, at least four, at least five, at least six, at least five, at least seven, at least eight, at least nine, at least ten, or more lymph nodes of the subject. In non-limiting embodiments, about 0.05 ml, about 0.1 ml, about 0.5 ml, about 1 ml, about 2 ml, or about 3 ml of thymic tissue can be delivered into a lymph node.

Non-limiting examples of lymph nodes in which the thymic tissue can be delivered to using methods disclosed herein include abdominal lymph nodes, celiac lymph nodes, paraaortic lymph nodes, splenic hilar lymph nodes, porta hepatis lymph nodes, left gastric lymph nodes, right gastric lymph nodes, left gastroomental (gastroepiploic) lymph nodes, right gastroomental (gastroepiploic) lymph nodes, retroperitoneal lymph nodes, pyloric lymph nodes (e.g., supra pyloric lymph nodes, sub pyloric lymph nodes, retro pyloric lymph nodes), pancreatic lymph nodes (e.g., superior pancreatic lymph nodes, inferior pancreatic lymph nodes, splenic lineal lymph nodes lymph nodes), splenic lymph nodes, hepatic lymph nodes (e.g., cystic lymph nodes, foraminal lymph nodes, foramen of Winslow), pancreaticoduodenal lymph nodes (e.g., superior pancreaticoduodenal lymph nodes, inferior pancreaticoduodenal lymph nodes), superior mesenteric lymph nodes, ileocolic lymph nodes, prececal lymph nodes, retrocecal lymph nodes, appendicular lymph nodes, mesocolic lymph nodes (e.g., paracolic lymph nodes, left colic lymph nodes, middle colic lymph nodes, right colic lymph nodes, inferior mesenteric lymph nodes, sigmoid lymph nodes, superior rectal lymph nodes), common iliac lymph nodes (e.g., medial common iliac lymph nodes, intermediate common iliac lymph nodes, lateral common iliac lymph nodes, subaortic common iliac lymph nodes, common iliac nodes of promontory), and external iliac lymph nodes (e.g., medial external iliac lymph nodes, intermediate external iliac lymph nodes, lateral external iliac lymph nodes, medial lacunar—femoral lymph nodes, intermediate lacunar—femoral lymph nodes, lateral lacunar—femoral lymph nodes, interiliac external iliac lymph nodes, obturator—external iliac obturatory lymph nodes).

In certain embodiments, it is important for the lymph node to be able to swell as the transplanted thymic tissue expands, and thus lymph nodes that are present in the peritoneal cavity can be particularly useful, especially, for example, where the lymph nodes are not closely associated with arteries or large veins.

### 6.2.3 Subject

In certain embodiments, the subject to be treated by the methods disclosed herein has received a thymectomy surgery. In certain embodiments, the subject to be treated by the methods disclosed herein is receiving a thymectomy surgery while the thymic tissue is delivered into the lymph node of the subject. In certain embodiments, the subject has a congenital heart defect and has received or is receiving an open heart surgery. Open heart surgery has become a routine method of treating critical congenital heart defects. Early cardiac surgical interventions for congenital heart defects can be associated with thymectomy, e.g., the partial or complete removal of the thymus. In certain embodiments, the thymectomy was performed in the subject during the open-heart surgery. In certain embodiments, the thymectomy was performed when the subject was a neonate. In certain embodiments, the thymectomy was performed when the subject was an infant. In certain embodiments, the thymectomy was performed when the subject was a child. In certain embodiments, the thymectomy was performed when the subject was at an age of at most 1 month, at most 2 months, at most 3 months, at most 4 months, at most 5 months, at most 6 months, at most 8 months, at most 10 months, at most 1 year, or at most 2 years. In certain embodiments, the thymectomy was performed when the subject was at the age of at most 1 month.

In some embodiments, a subject to be treated by compositions and methods of the disclosure has a condition that affects the thymus. Non limiting examples of conditions that affect the thymus include myasthenia gravis, pure red cell aplasia, hypogammaglobulinemia, thymus cancer, thymoma, type A thymoma, type B thymoma, autoimmune diseases, T cell-mediated autoimmunity, T cell lymphopenia, thymic atrophy, age-related thymic atrophy, thymic cyst, thymic hyperplasia, thymic hypoplasia, thymic aplasia, thymic dysplasia, severe combined immunodeficiency, Nezelof syndrome, Wiscott-Aldrich syndrome DiGeorge syndrome, recurrent infection, recurrent viral infection, premature immunologic aging, and cancer.

Subjects to be treated by the methods disclosed herein can be of any age. In certain embodiments, the subject to be treated by the methods disclosed herein is a neonate, an infant, a child, an adolescent, or an adult.

In certain embodiments, the subject to be treated by the methods disclosed herein is a human neonate that has attained the age of at most about 1 month. In certain embodiments, the subject to be treated by the methods disclosed herein is a human infant that has attained an age of between about 1 month and about 2 years. In certain embodiments, the subject to be treated by the methods disclosed herein is a human child that has attained an age between about 2 years and about 12 years. In certain embodiments, the subject to be treated by the methods disclosed herein is a human adolescent that has attained an age between about 12 years and about 18 years. In certain embodiments, the subject to be treated by the methods disclosed herein is a human adult that has attained an age of at least about 18 years. In certain embodiments, the subject to be treated is a middle aged or older human, for example, with an age-related decline in thymic function.

In certain embodiments, the subject to be treated by the methods disclosed herein is a human subject that has attained an age of at most about 1 month, at most about 3 months, at most about 6 months, at most about 1 year, at most about 2 years, at most about 3 years, at most about 4 years, at most about 5 years, at most about 6 years, at most about 7 years, at most about 8 years, at most about 9 years, at most about 10 years, at most about 11 years, at most about 12 years, at most about 13 years, at most about 14 years, at most about 15 years, at most about 16 years, at most about 17 years, at most about 18 years, or at least 18 years.

In certain embodiments, the subject to be treated by the methods disclosed herein is a human subject that has attained an age of at least about 1 month, at least about 3 months, at least about 6 months, at least about 1 year, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years, at least about 16 years, at least about 17 years, at least about 18 years, at least about 25 years, at least about 30 years, at least about 35 years, at least about 40 years, at least about 45 years, at least about 50 years, at least about 55 years, at least about 60 years, at least about 65 years, at least about 70 years, or at least about 75 years.

In certain embodiments, the thymic tissue was obtained from the subject when the subject was a neonate, an infant, or a child and was cryopreserved before later being thawed and delivered to the subject. In certain embodiments, the cryopreserved thymic tissue can be delivered to the subject after the thymectomy and at any age of the subject. In certain embodiments, the thymic tissue obtained from the subject can be cryopreserved in multiple vials, and each vial contains a single dose of the thymic tissue that can be delivered to the subject. In certain embodiments, the cryopreserved thymic tissue is delivered to the subject in multiple doses over the lifetime of the subject.

### 6.3 Kits

The present disclosure provides kits for preserving, restoring, or increasing thymic functions of a subject, e.g., a subject that has received or is receiving a thymectomy surgery. In certain embodiments, the kits include thymic tissue and tools for delivery of the thymic tissue to the subject, and the thymic tissue is autologous to the subject. In certain embodiments, the thymic tissue is provided in a solution included in the kits.

In certain embodiments, the solution includes different types of cells that constitute the thymic tissue. In certain embodiments, the solution further includes pharmaceutically acceptable excipients, diluents, or carriers. Pharmaceutically acceptable carriers and diluents include saline, aqueous buffer solutions, solvents, and/or dispersion media. Non-limiting examples of pharmaceutically acceptable excipients that can be used with the presently disclosed kits include sugars, such as lactose, glucose and sucrose; starches, such as corn starch and potato starch; cellulose, and its derivatives, such as sodium carboxymethyl cellulose, methylcellulose, ethyl cellulose, microcrystalline cellulose and cellulose acetate; powdered tragacanth; malt; gelatin; lubricating agents, such as magnesium stearate, sodium lauryl sulfate and talc; cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol (PEG); esters, such as ethyl oleate and ethyl laurate; agar; buffering agents, such as magnesium hydroxide and aluminum hydroxide; alginic acid; pyrogen free water; isotonic saline; Ringer's solution; ethyl alcohol; pH buffered solutions; polyesters, polycarbonates and/or polyanhydrides; bulking agents, such as polypeptides and amino acids; serum component, such as serum albumin, HDL and LDL; C2-C12 alcohols, such as ethanol; and other non-toxic compatible substances employed in pharmaceutical formulations.

In certain embodiments, the thymic tissue included in the kits is cryopreserved. In certain embodiments, the thymic tissue included in the kit is cryopreserved for about 12 hours, about 24 hours, about 2 days, about 3 days, about 1 week, about 2 weeks, about 3 weeks, about 4 weeks, about 2 months, about 3 months, about 4 months, about 5 months, about 6 months, about 8 months, about 10 months, about 1 year, about 2 years, about 3 years, about 4 years, about 5 years, about 6 years, about 7 years, about 8 years, about 9 years, about 10 years, about 15 years, about 20 years, or more. In certain embodiments, the thymic tissue included in the kit is cryopreserved for at least about 12 hours, at least about 24 hours, at least about 2 days, at least about 3 days, at least about 1 week, at least about 2 weeks, at least about 3 weeks, at least about 4 weeks, at least about 2 months, at least about 3 months, at least about 4 months, at least about 5 months, at least about 6 months, at least about 8 months, at least about 10 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In certain embodiments, the thymic tissue included in the kit is cryopreserved for at most about 12 hours, at most about 24 hours, at most about 2 days, at most about 3 days, at most about 1 week, at most about 2 weeks, at most about 3 weeks, at most about 4 weeks, at most about 2 months, at most about 3 months, at most about 4 months, at most about 5 months, at most about 6 months, at most about 8 months, at most about 10 months, at most about 1 year, at most about 2 years, at most about 3 years, at most about 4 years, at most about 5 years, at most about 6 years, at most about 7 years, at most about 8 years, at most about 9 years, at most about 10 years, at most about 15 years, at most about 20 years, at most about 30 years, at most about 40 years, at most about 50 years, or less.

In certain embodiments, the thymic tissue included in the kits is in an amount of at least about 0.1 gram, at least about 0.2 gram, at least about 0.3 gram, at least about 0.4 gram, at least about 0.5 gram, at least about 0.6 gram, at least about 0.7 gram, at least about 0.8 gram, at least about 0.9 gram, at least about 1 gram, at least about 1.5 grams, at least about 2 grams, at least about 2.5 grams, at least about 3 grams, at least about 4 grams, at least about 5 grams, at least about 6 grams, at least about 7 grams, at least about 8 grams, at least about 9 grams, or at least about 10 grams. In certain embodiments, the thymic tissue included in the kits is in an amount of up to about 5 grams, up to about 10 grams, up to about 15 grams, or up to about 20 grams. In certain embodiments, the thymic tissue included in the kits is in an amount of about 0.1 gram, about 0.2 gram, about 0.3 gram, about 0.4 gram, about 0.5 gram, about 0.6 gram, about 0.7 gram, about 0.8 gram, about 0.9 gram, about 1 gram, about 2 grams, about 3 grams, about 4 grams, about 5 grams, about 6 grams, about 8 grams, about 10 grams, about 13 grams, about 15 grams, about 18 grams, about 20 grams, or more.

In certain embodiments, the thymic tissue included in the kits is in a size of at least about 0.1 cm³, at least about 0.2 cm³, at least about 0.3 cm³, at least about 0.4 cm³, at least about 0.5 cm³, at least about 0.6 cm³, at least about 0.7 cm³, at least about 0.8 cm³, at least about 0.9 cm³, at least about 1 cm³, at least about 1.5 cm³, at least about 2 cm³, at least about 2.5 cm³, at least about 3 cm³ at least about 4 cm³, or at least about 5 cm³. In certain embodiments, the thymic tissue included in the kits is in a size of up to about 5 cm³, up to about 10 cm³, up to about 15 cm³, or up to about 20 cm³. In certain embodiments, the thymic tissue included in the kits is in a size of about 0.1 cm³, about 0.2 cm³, about 0.3 cm³, about 0.4 cm³, about 0.5 cm³, about 0.6 cm³, about 0.7 cm³, about 0.8 cm³, about 0.9 cm³, about 1 cm³, about 2 cm³, about 3 cm³, about 4 cm³, about 5 cm³, about 6 cm³, about 8 cm³, about 10 cm³, about 13 cm³, about 15 cm³, about 18 cm³, about 20 cm³, or more.

In certain embodiments, the thymic tissue included in the kits is in an amount effective to restore the thymic function of the subject. In certain embodiments, the thymic tissue included in the kits is in an amount effective to expand in the lymph node, where the expanded thymic tissue restores the thymic function of the subject. In certain embodiments, the thymic tissue included in the kits is in an amount effective to increase the thymic function of the subject. In certain embodiments, the thymic tissue included in the kits is in an amount effective to engraft into the lymph node. In certain embodiments, the thymic tissue included in the kits is in an amount effective to form an ectopic thymus tissue in the lymph node. In certain embodiments, the thymic tissue included in the kits is in an amount effective to increase a level of circulating T cells in the subject (e.g., circulating naïve T cells). In certain embodiments, the thymic tissue included in the kits is in an amount effective to increase the diversity of circulating T cells in the subject (e.g., increase the repertoire of TCRs and recognized cognate antigens).

In some embodiments, a kit of the disclosure can include suitable components for harvesting a thymic tissue, processing a thymic tissue, storing (e.g., cryopreserving) a thymic tissue, culturing a thymic tissue, delivering a thymic tissue to a subject, or a combination thereof In certain embodiments, the kits do not include a thymic tissue.

In certain embodiments, the kit contains materials necessary to cryopreserve a thymic tissue, for example, reagents and/or containers. In certain embodiments, the kit is for cryopreserving the thymic tissue for about 12 hours, about 24 hours, about 2 days, about 3 days, about 1 week, about 2 weeks, about 3 weeks, about 4 weeks, about 2 months, about 3 months, about 4 months, about 5 months, about 6 months, about 8 months, about 10 months, about 1 year, about 2 years, about 3 years, about 4 years, about 5 years, about 6 years, about 7 years, about 8 years, about 9 years, about 10 years, about 15 years, about 20 years, or more. In certain embodiments, the kit is for cryopreserving the thymic tissue for at least about 12 hours, at least about 24 hours, at least about 2 days, at least about 3 days, at least about 1 week, at least about 2 weeks, at least about 3 weeks, at least about 4 weeks, at least about 2 months, at least about 3 months, at least about 4 months, at least about 5 months, at least about 6 months, at least about 8 months, at least about 10 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In certain embodiments, the kit is for cryopreserving the thymic tissue for at most about 12 hours, at most about 24 hours, at most about 2 days, at most about 3 days, at most about 1 week, at most about 2 weeks, at most about 3 weeks, at most about 4 weeks, at most about 2 months, at most about 3 months, at most about 4 months, at most about 5 months, at most about 6 months, at most about 8 months, at most about 10 months, at most about 1 year, at most about 2 years, at most about 3 years, at most about 4 years, at most about 5 years, at most about 6 years, at most about 7 years, at most about 8 years, at most about 9 years, at most about 10 years, at most about 15 years, at most about 20 years, at most about 30 years, at most about 40 years, at most about 50 years, or less.

In certain embodiments, the kit is for harvesting, processing, storing, culturing, and/or delivering an amount of thymic tissue that is at least about 0.1 gram, at least about 0.2 gram, at least about 0.3 gram, at least about 0.4 gram, at least about 0.5 gram, at least about 0.6 gram, at least about 0.7 gram, at least about 0.8 gram, at least about 0.9 gram, at least about 1 gram, at least about 1.5 grams, at least about 2 grams, at least about 2.5 grams, at least about 3 grams, at least about 4 grams, at least about 5 grams, at least about 6 grams, at least about 7 grams, at least about 8 grams, at least about 9 grams, or at least about 10 grams.

In certain embodiments, the kit is for harvesting, processing, storing, culturing, and/or delivering an amount of thymic tissue that is up to about 5 grams, up to about 10 grams, up to about 15 grams, or up to about 20 grams. In certain embodiments, the kit is for harvesting, processing, storing, culturing, and/or delivering an amount of thymic tissue that is about 0.1 gram, about 0.2 gram, about 0.3 gram, about 0.4 gram, about 0.5 gram, about 0.6 gram, about 0.7 gram, about 0.8 gram, about 0.9 gram, about 1 gram, about 2 grams, about 3 grams, about 4 grams, about 5 grams, about 6 grams, about 8 grams, about 10 grams, about 13 grams, about 15 grams, about 18 grams, about 20 grams, or more.

In certain embodiments, the kit is for harvesting, processing, storing, culturing, and/or delivering a thymic tissue that has a size of at least about 0.1 cm³, at least about 0.2 cm³, at least about 0.3 cm³, at least about 0.4 cm³, at least about 0.5 cm³, at least about 0.6 cm³, at least about 0.7 cm³, at least about 0.8 cm³, at least about 0.9 cm³, at least about 1 cm³, at least about 1.5 cm³, at least about 2 cm³, at least about 2.5 cm³, at least about 3 cm³ at least about 4 cm³, or at least about 5 cm³. In certain embodiments, the kit is for harvesting, processing, storing, culturing, and/or delivering a thymic tissue that has a size of up to about 5 cm³, up to about 10 cm³, up to about 15 cm³, or up to about 20 cm³. In certain embodiments, the kit is for harvesting, processing, storing, culturing, and/or delivering a thymic tissue that has a size of about 0.1 cm³, about 0.2 cm³, about 0.3 cm³, about 0.4 cm³, about 0.5 cm³, about 0.6 cm³, about 0.7 cm³, about 0.8 cm³, about 0.9 cm³, about 1 cm³, about 2 cm³, about 3 cm³, about 4 cm³, about 5 cm³, about 6 cm³, about 8 cm³, about 10 cm³, about 13 cm³, about 15 cm³, about 18 cm³, about 20 cm³, or more.

In some embodiments, the kit is for delivering a thymic tissue in a volume of at least about 0.1 mL, at least about 0.2 mL, at least about 0.3 mL, at least about 0.4 mL, at least about 0.5 mL, at least about 0.6 mL, at least about 0.7 mL, at least about 0.8 mL, at least about 0.9 mL, at least about 1 mL, at least about 1.5 mL, at least about 2 mL, at least about 2.5 mL, at least about 3 mL, at least about 4 mL, at least about 5 mL, at least about 6 mL, at least about 7 mL, at least about 8 mL, at least about 9 mL, or at least about 10 mL (for example, per lymph node, or in total split between two or more lymph nodes). In some embodiments, the kit is for delivering a thymic tissue in a volume of up to about 5 mL, up to about 10 mL, up to about 15 mL, or up to about 20 mL (for example, split between two or more lymph nodes). In some embodiments, the kit is for delivering a thymic tissue in a volume of about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 2 mL, or about 3 mL per lymph node. In some embodiments, the kit is for delivering a thymic tissue in a volume of about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 8 mL, about 10 mL, about 13 mL, about 15 mL, about 18 mL, about 20 mL, or more, split between two or more lymph nodes.

In some embodiments, the kit is for delivering a thymic tissue to a subject in an amount that is relative to the subject's body weight, for example, at least about 0.001 mg/kg, at least about 0.005 mg/kg, at least about 0.01 mg/kg, at least about 0.05 mg/kg, at least about 0.1 mg/kg, at least about 0.5 mg/kg, at least about 1 mg/kg, at least about 5 mg/kg, at least about 10 mg/kg, at least about 15 mg/kg, at least about 20 mg/kg, at least about 25 mg/kg, at least about 30 mg/kg, at least about 40 mg/kg, at least about 50 mg/kg, at least about 60 mg/kg, at least about 70 mg/kg, at least about 80 mg/kg, at least about 90 mg/kg, at least about 100 mg/kg, at least about 110 mg/kg, at least about 120 mg/kg, at least about 130 mg/kg, at least about 140 mg/kg, at least about 150 mg/kg, at least about 160 mg/kg, at least about 170 mg/kg, at least about 180 mg/kg, at least about 190 mg/kg, at least about 200 mg/kg, at least about 250 mg/kg, at least about 500 mg/kg, at least about 1000 mg/kg, or at least about 1500 mg/kg. In some embodiments, the amount is at most about 1 mg/kg, at most about 10 mg/kg, at most about 20 mg/kg, at most about 50 mg/kg, at most about 100 mg/kg, at most about 110 mg/kg, at most about 120 mg/kg, at most about 130 mg/kg, at most about 140 mg/kg, at most about 150 mg/kg, at most about 160 mg/kg, at most about 170 mg/kg, at most about 180 mg/kg, at most about 190 mg/kg, at most about 200 mg/kg, at most about 250 mg/kg, at most about 500 mg/kg, at most about 1000 mg/kg, or at most about 1500 mg/kg. In some embodiments, the amount is about 0.001 mg/kg, about 0.005 mg/kg, about 0.01 mg/kg, about 0.05 mg/kg, about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 250 mg/kg, about 500 mg/kg, about 1000 mg/kg, or about 1500 mg/kg.

In certain embodiments, the kit is for harvesting, processing, storing, culturing, and/or delivering a thymic tissue in an amount effective to restore the thymic function of the subject. In certain embodiments, the kit is for harvesting, processing, storing, culturing, and/or delivering a thymic tissue in an amount effective to expand in the lymph node, e.g., where the expanded thymic tissue restores the thymic function of the subject. In certain embodiments, the kit is for harvesting, processing, storing, culturing, and/or delivering a thymic tissue in an amount effective to increase the thymic function of the subject. In certain embodiments, the kit is for harvesting, processing, storing, culturing, and/or delivering a thymic tissue in an amount effective to engraft into the lymph node. In certain embodiments, the kit is for harvesting, processing, storing, culturing, and/or delivering a thymic tissue in an amount effective to form an ectopic thymus tissue in the lymph node. In certain embodiments, the kit is for harvesting, processing, storing, culturing, and/or delivering a thymic tissue in an amount effective to increase a level of circulating T cells in the subject (e.g., circulating naïve T cells).

In certain embodiments, the kits further include instructions for preserving, increasing, or restoring thymic functions of a subject that has received or is receiving a thymectomy surgery. In certain embodiments, the instructions include methods, for example, as described herein, e.g., in Section 6.2 of the present disclosure.

Any suitable tools known in the art for the delivery of the thymic tissue to the lymph node of the subject can be included in the kits disclosed herein. Non-limiting examples of tools for delivery of the thymic tissue include tubes, syringes, needles (e.g., microinjection glass needles), containers suitable for cryopreservation, containers suitable for tissue culture, and any tools that are required for minimally invasive procedures.

Although the presently disclosed subject matter and certain of its advantages have been described in detail, it should be understood that various changes, substitutions, and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which is incorporated herein by reference in their entireties for all purposes.

### 7. EXAMPLES

The presently disclosed subject matter will be better understood by reference to the following Examples, which are provided as exemplary of the presently disclosed subject matter, and not by way of limitation.

### 7.1 Example 1: Nude Mouse Dosage Study

Nude mice lack a thymus and therefore are unable to produce T cells. In consequence, nude mice are immunodeficient. In these mice, a functional immune system is generated by thymus transplantation in a mesenteric lymph node. Here, the question of the amount of thymic tissue necessary to regenerate the presence of blood T-cells, and if the amount of thymic tissue transplanted into the lymph node can yield a sufficient number of circulating blood T cells are addressed.

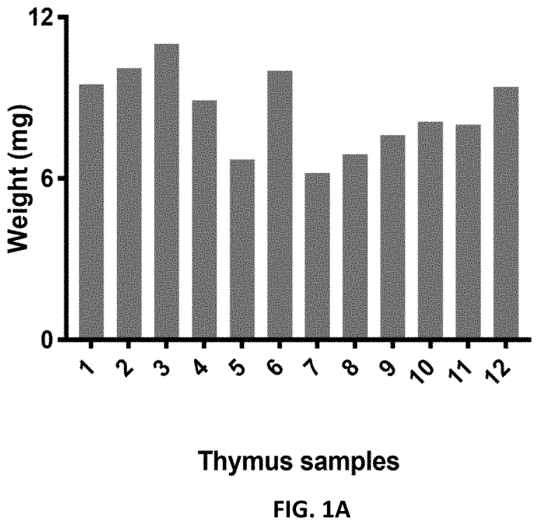

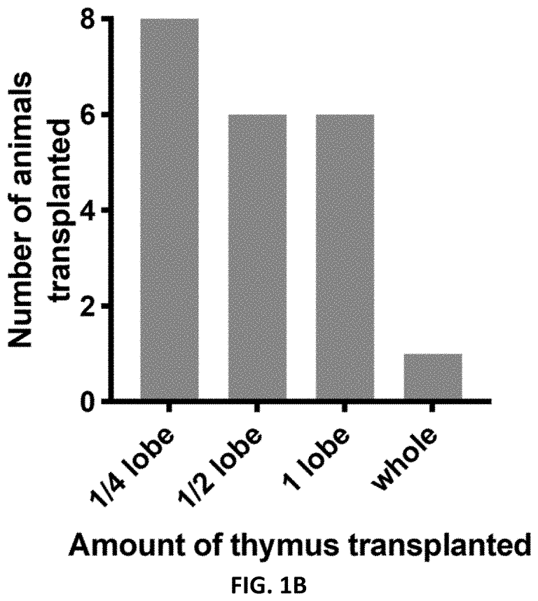

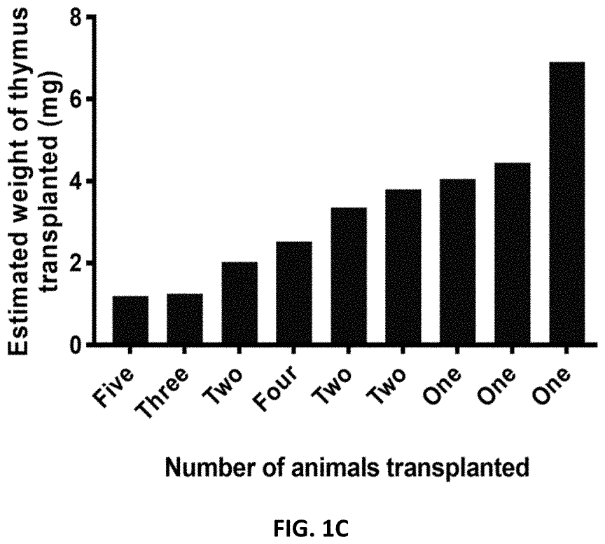

Twenty-three 15-week-old BALB/c nude mice were enrolled (n=16 females; n=7 males). Twenty-one mice received transplantation of ¼ of a lobe to 2 lobes of BALB/c wild type newborn thymus in their lymph nodes (2 thymic lobes=1 full thymus). The remaining two mice received transplantation of thymic single-cell suspension derived from the same donor mice. Samples to be transplanted were retrieved from 12 different thymi of 2 litters and weighing, on average 8.5 mg (range 6.2-11 mg; FIG. 1A). Eight mice received transplantation of ¼ of thymus lobe, 6 mice received either ½ or an entire thymus lobe, and one mouse received an entire thymus (2 lobes) (FIG. 1B). When ½ lobe dose was used, the tissue was further fragmented into two pieces for transplantation. For the 1-lobe dose, 4 total fragments were transplanted in the lymph node, while for the 2-lobe dose, 8 total fragments were transplanted. This approach was taken because of the small size of the nude mouse lymph nodes (just above 1 mm3 on average), which limits the feasibility of tissue fragments transplantation. FIG. 1C shows the estimated weights of thymus fragments transplanted in animals.

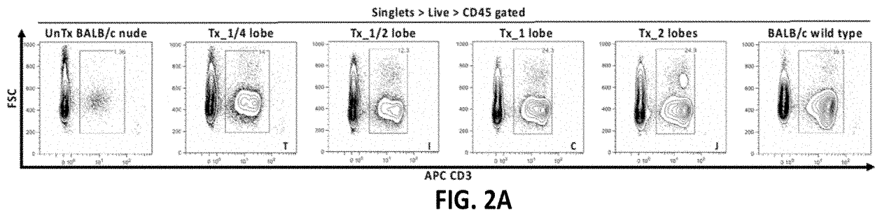

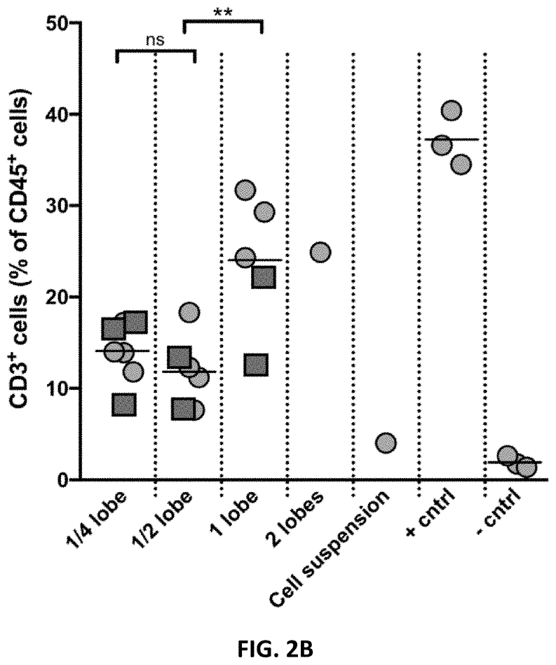

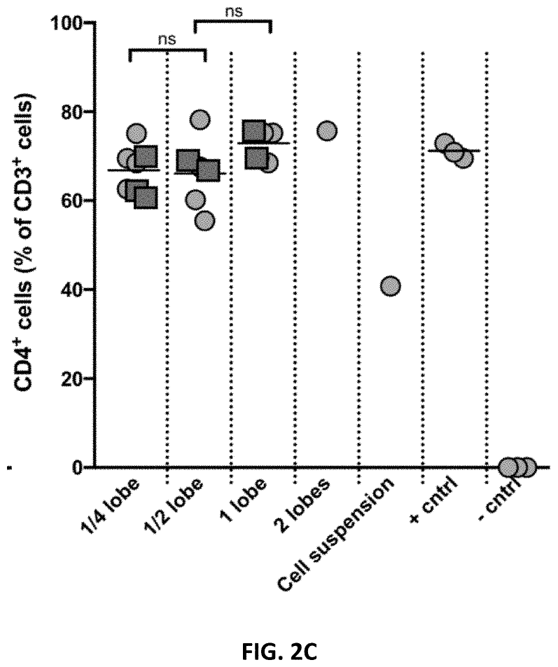

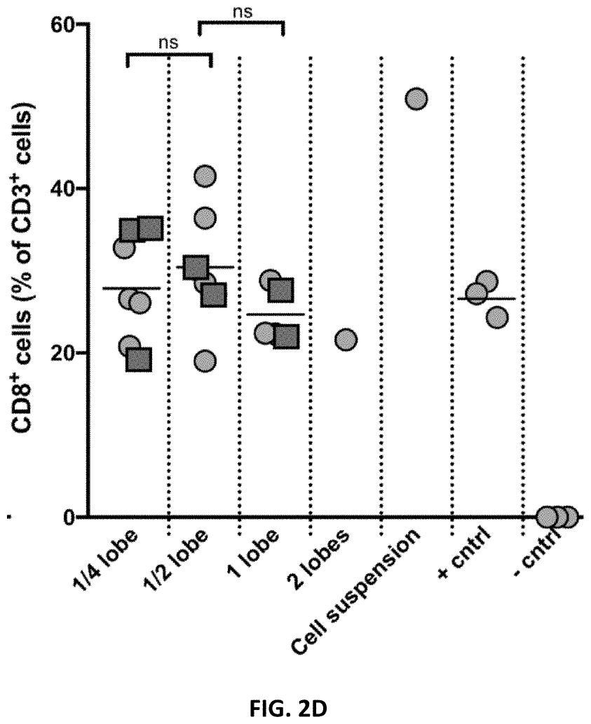

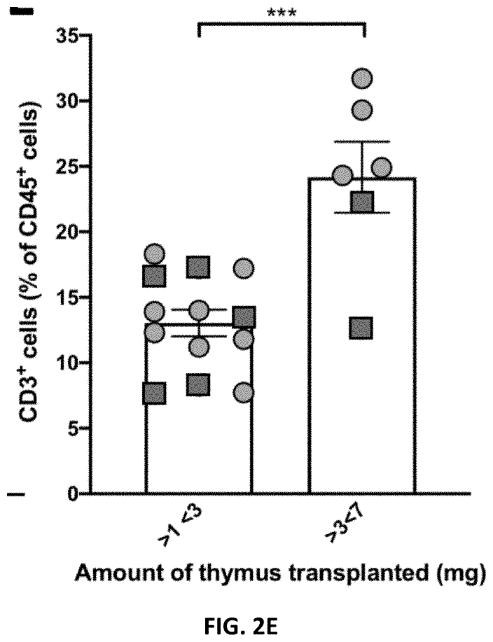

Four months after transplantation, 20 surviving mice were analyzed by flow cytometry for the presence of T cells in the blood. Three BALB/c nude mice, not transplanted, and three BALB/c wild type mice were used as negative and positive controls for circulating blood T-cells, respectively. Using flow cytometric analyses, FIG. 2A shows representative dot plots of CD3 expression in different conditions. Cells were first gated for singlets, followed by live cells, CD45+ cells, and finally, for CD3 expression. Letters indicate mouse ID. Percentages of CD3+ T cells for each mouse are given in FIG. 2B, where circular dots indicate females, and square dots, males. All groups that received at least ¼ of a thymic lobe or more exhibited an increased proportion of circulating CD3+ T cells compared to controls that did not receive a transplant. Mice transplanted with either ¼ or ½ lobe doses showed comparable CD3+ T cell percentages (14.13±3.29 and 11.76±3.98, respectively; P=0.2634). CD3+ T cell percentages were significantly higher when 1 lobe of the thymus was transplanted compared to half a lobe (24.02±7.43;P=0.0067 for ½ lobe versus 1 lobe group). Transplantation of 2 lobes generated similar CD3+ T cell percentages to the 1 lobe dose, while transplantation of a thymic cell suspension did not significantly increase the frequency of circulating T cells. Regardless of the amount of tissue transplanted, a normal CD4/CD8 ratio could be achieved (FIG. 2C and 2D). Each dot represents a mouse. Circular dots indicate females, and square dots indicate males.

CD3+ T cell percentages in transplanted mice correlate with the actual weight (mg) of thymus tissue transplanted. The transplanted mice were divided into 2 groups, with one group including mice that had received from 1 mg and up to under 3 mg of thymus tissue, and the other group including mice that had received over 3 mg and up to 7 mg of thymus tissue. There was a statistically significant difference in the production of CD3+ blood T-cells between these two groups (P=0.0002; FIG. 2E). In this analysis, mice were divided into 2 groups based on the actual weight (mg) of thymus transplanted in their lymph nodes instead of thymic lobe fractions. *** indicates P-value is less than 0.001. ** indicates that P-value is less than 0.01. ns indicates “not significant” (i.e., P-value is more than 0.05).

These results suggest two conclusions: (1) a dose-response exists between the weight of thymus tissue transplanted in the lymph node and the percentage of circulating T cells in the transplanted mice. (2) a threshold is present in the weight of thymic tissue transplanted in the lymph node to obtain a higher number of circulating T-cells closer to wild-type, and possibly improved T-cell function.

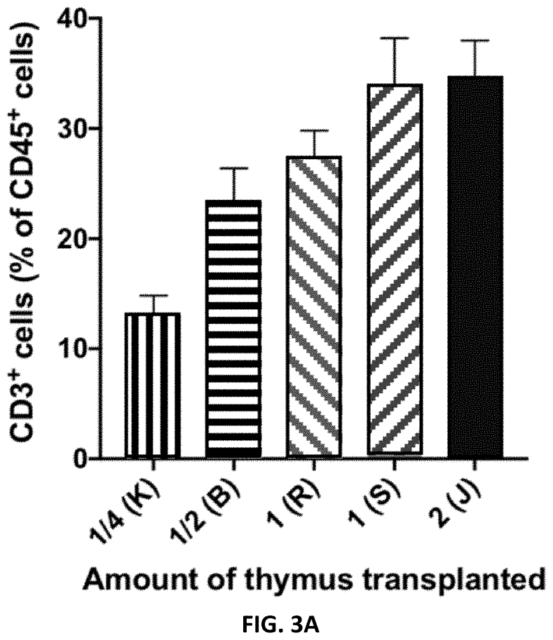

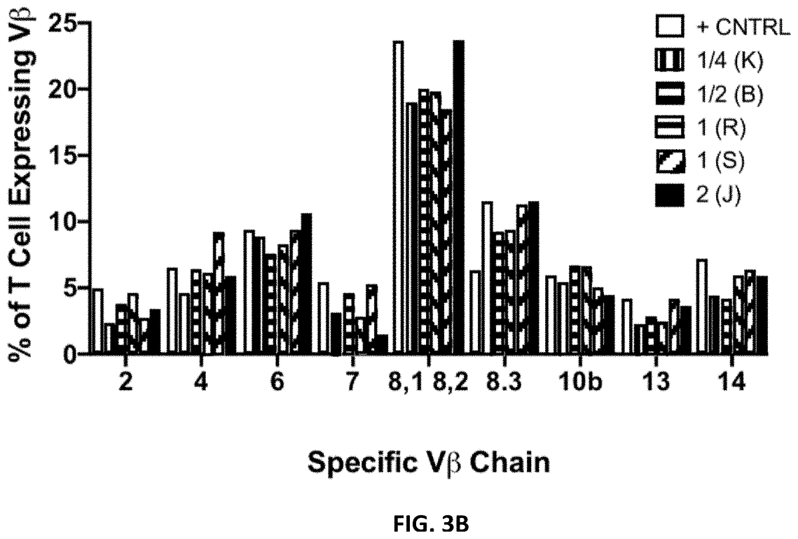

One month later, the T cell receptor (TCR) Vβ repertoire of circulating blood T cells in 5 transplanted mice was analyzed by flow cytometry using monoclonal antibodies directed against 15 different variants. CD3+ T cell percentages obtained at this time point were comparable to those obtained one month earlier, with the only exception for the mouse transplanted with an entire thymus, whose T cell percentages had slightly increased (FIG. 3A). Means were obtained from 16 different measurements. 9 TCR-Vβ variants were identified to be expressed in both transplanted mice and one control BALB/c wild type mouse (FIG. 3B). A BALB/c wild type mouse was included as a positive control (CNTRL). No differences were observed among the different conditions (one-way ANOVA P=0.9956), demonstrating that delivery of thymic tissue into a lymph node can increase circulating T cell diversity in recipients that lack a thymus or have a functionally-deficient thymus.

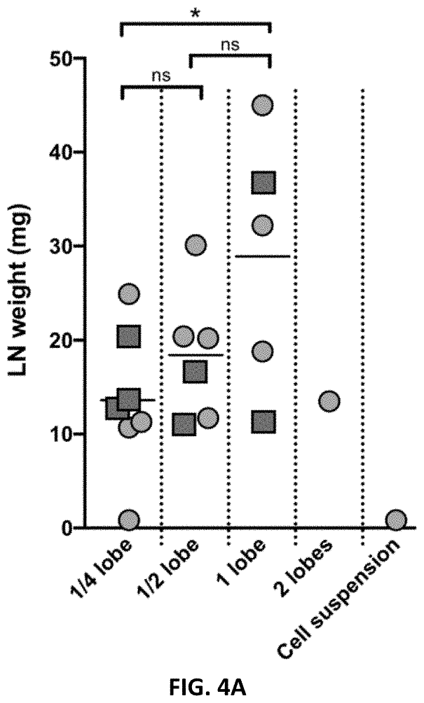

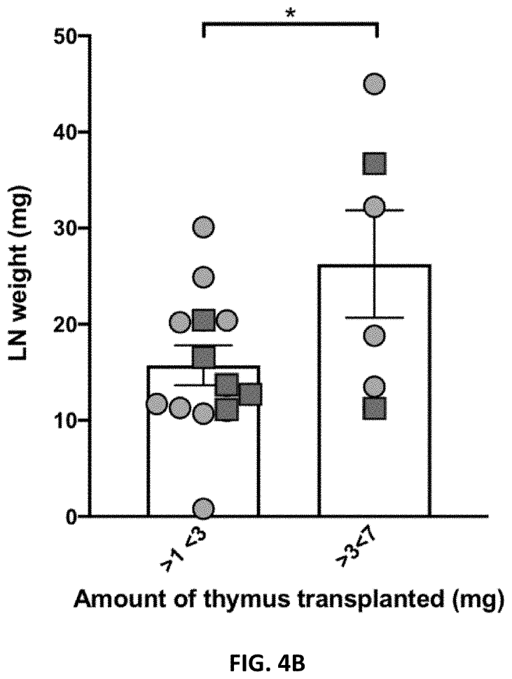

All 20 mice were terminated 6 months and a half after transplantation. FIG. 4A shows the weights of isolated lymph nodes.

Similarly to FIG. 2B, the weight of lymph nodes transplanted with ¼ thymus lobe was similar to the weight of lymph nodes transplanted with ½ thymus lobe (13.49±7.65 and 18.33±7.03, respectively; P=0.2628; FIG. 4A). Each dot represents a mouse. Circular dots indicate females; square dots indicate males. Lymph node weights were significantly higher when 1 lobe of the thymus was transplanted (28.8±13.62; P=0.0315 for ¼ lobe versus 1 lobe group). In the same way as for data in FIG. 2E, the isolated lymph nodes were divided into 2 groups, based on the weight (mg) of thymus tissue they had received. Again, there was a statistically significant difference between these two groups (P=0.0428; FIG. 4B). * indicates that the P-value is less than 00.5.

The disclosed data suggests a relationship between the weight of thymus tissue transplanted in the lymph node, the weight of the lymph node at the time of sacrifice, and the number of circulating blood T cells in transplanted mice. These results suggest that, in subjects where size limitations exist, more lymph nodes can be transplanted in order to obtain a higher level of circulating blood T cells, for example, a percentage close to that of a subject with a normal functioning thymus. The results also suggest that females can be more effective in producing new T cells after thymus transplantation in this model. Without wishing to be bound by theory, gender differences in response to thymus transplantation may be a consequence of hormonal differences between females and males in this model.

The above results indicate that transplanting more thymus weight can be associated with a higher level of naïve T cell reconstitution. For example, in some embodiments, transplanting at least a third of a full thymus weight can result in a higher level of naïve T cell reconstitution compared to transplanting less than a third of a full thymus weight. For example, over 3 mg of an average 8.5 mg full-size newborn mouse thymus can translate in a human patient of just under 9 gr of a 25 gr full-size pediatric thymus. Considering that in some cases around 1 ml is the maximum volume that can be injected into human lymph nodes, a total of 9 lymph nodes (1 gr thymic tissue per lymph node) can be injected in lymph nodes to obtain ⅓ of a full pediatric thymus.

Thymectomy can result in T-cell lymphopenia, characterized by a decrease in naïve T cells with a concomitant increase in the memory T-cell populations. Furthermore, certain patients undergoing a thymectomy (e.g., early in life, such as <6 months with >90% thymic removal) show a quantitative defect in the T-cell compartment later in life, primarily affecting the naive T-cell population.

If these patients are auto-transplanted with thymic tissues according to compositions and methods of the disclosure, blood circulating CD3+ T cells can increase and particularly the naïve T-cell population can be restored.

### 7.2 Example 2: Aging Wild Type Mouse Study

This Example address whether transplantation of a low dose of the thymus, ¼ of a thymus lobe in the lymph node, can rejuvenate immune functions in transplanted mice and whether there are any differences between females and males.

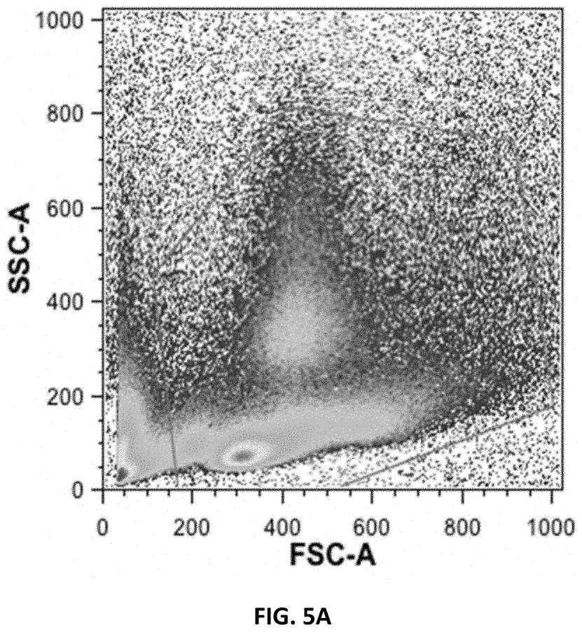

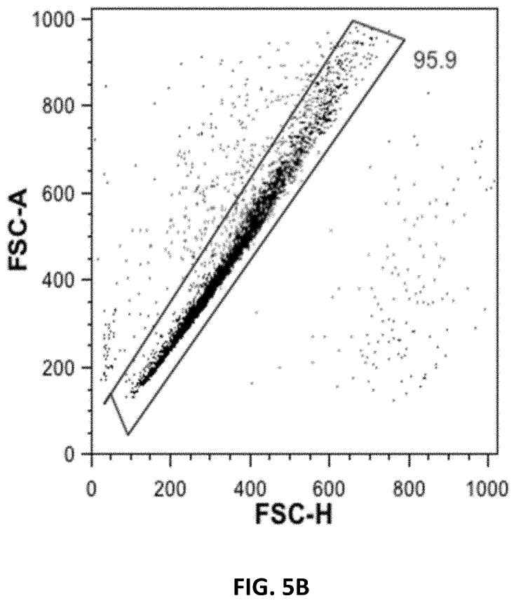

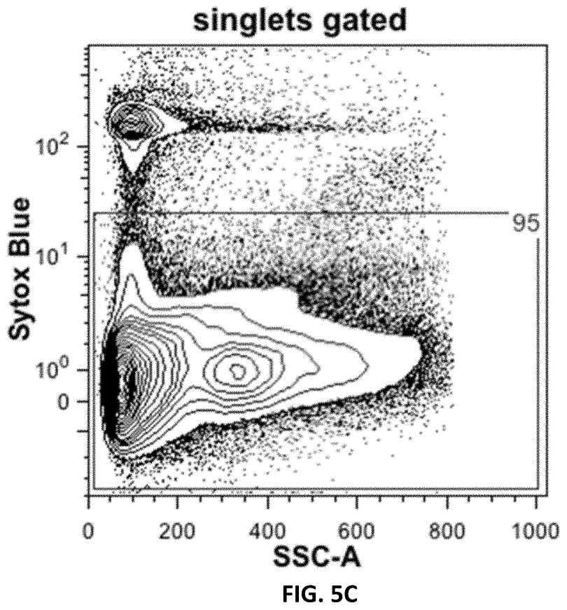

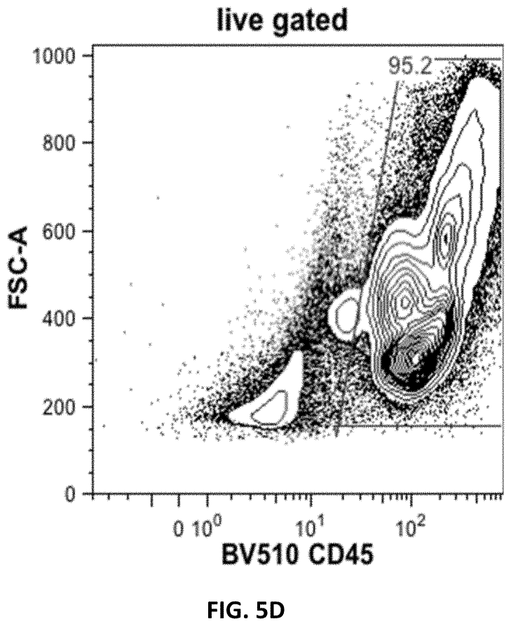

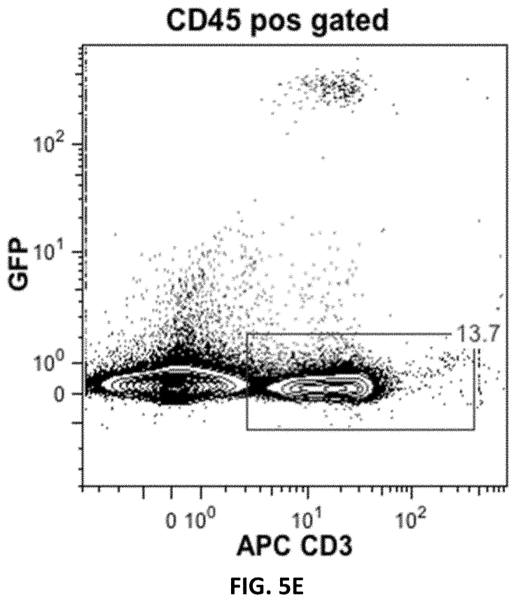

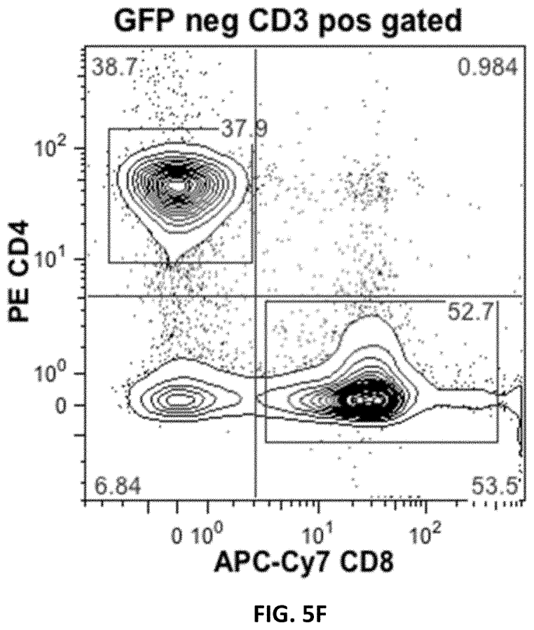

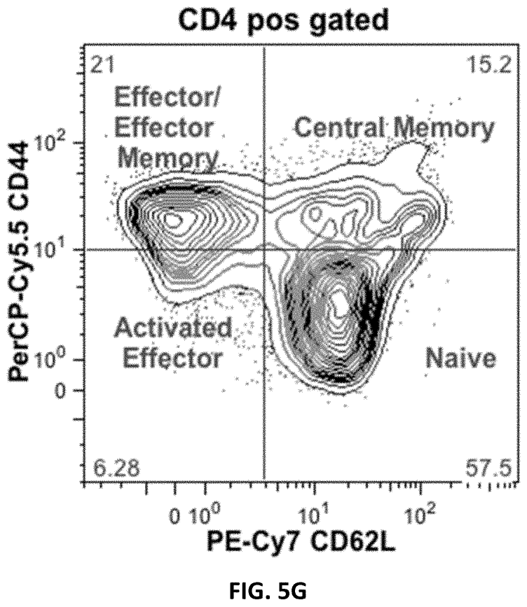

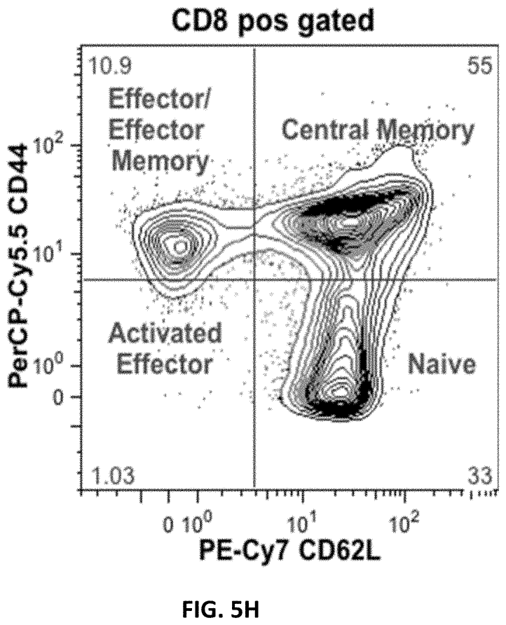

Thirty-two C57BL/6J mice, 16 females and 16 males were enrolled. Each mouse group, in turn, comprised 8 adult mice (18-week-old) and 8 old mice (62-week-old). Half of the mice in each group were transplanted with ¼ thymus lobe. Donor thymi were isolated from GFP+ C57BL/6J newborn mice from the same litter. Seven weeks after thymus transplantation, all 32 mice were analyzed by flow cytometry for naïve, effector, and memory T cells, exploiting the differential expression of CD44 and CD62L in these subpopulations (FIGS. 5A-5H). The gating strategy is provided in FIGS. 5A-5H. FIG. 5A illustrates a forward scatter vs side scatter gate. FIG. 5B illustrates a gate for single cells. FIG. 5C illustrates a gate for live cells. FIG. 5D illustrates a gate for CD45+ cells. FIG. 5E illustrates a gate for CD3+ cells. FIG. 5F illustrates gates for CD4+ and CD8+ cells. FIG. 5G illustrates gating of CD4+ cells for naïve, activated effector, effector memory, and central memory cells. FIG. 5H illustrates gating of CD8+ cells for naïve, activated effector, effector memory, and central memory cells.

Naïve T cells are continually generated in the thymus, but as the mouse ages and the thymus involutes, the proportion of naïve CD4+ and CD8+ T cells reduces. Therefore, whether thymus transplantation in an old animal could increase populations of naïve T cells was tested.

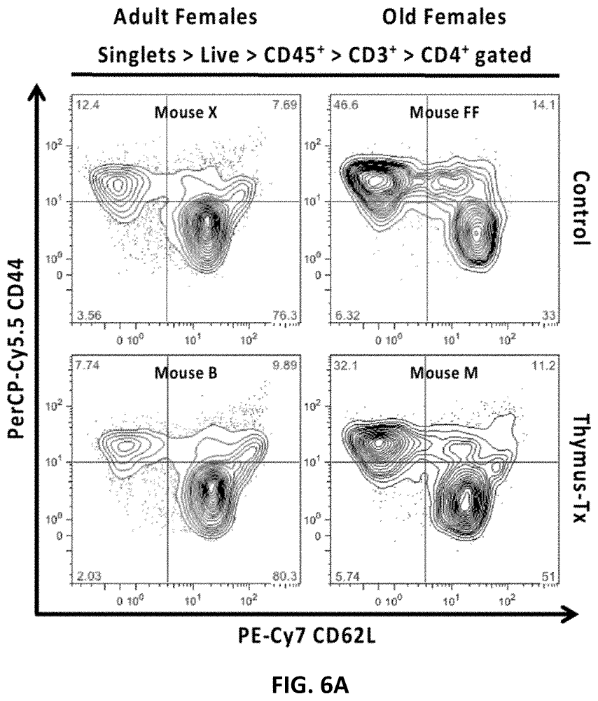

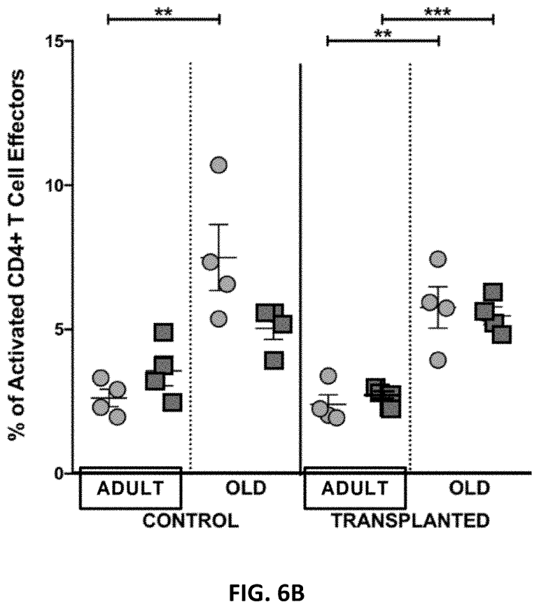

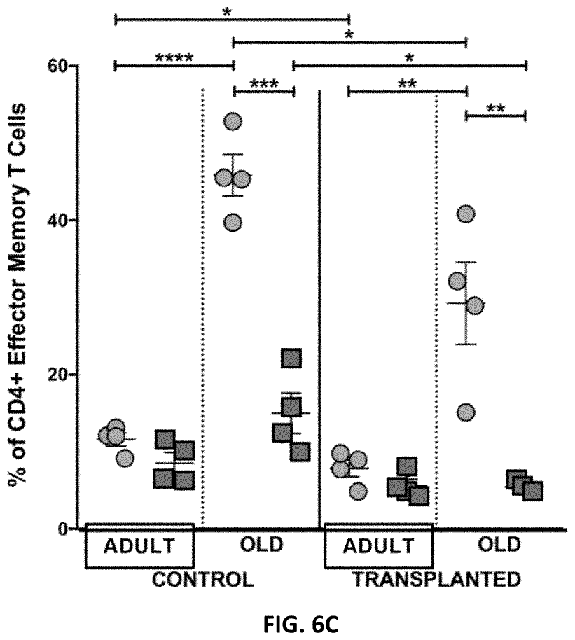

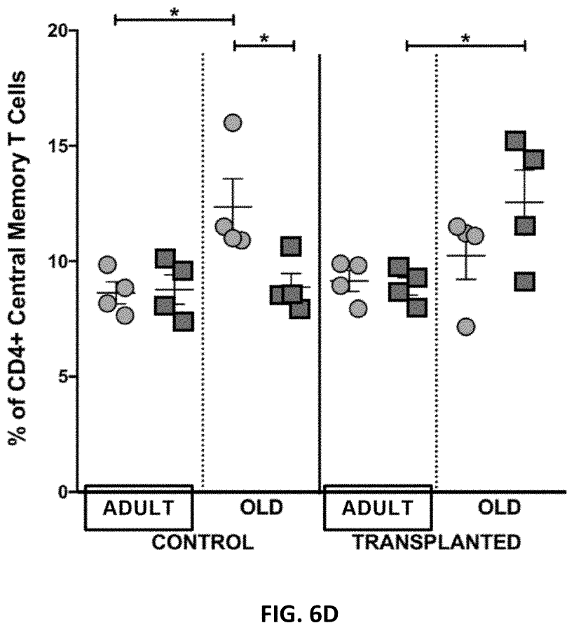

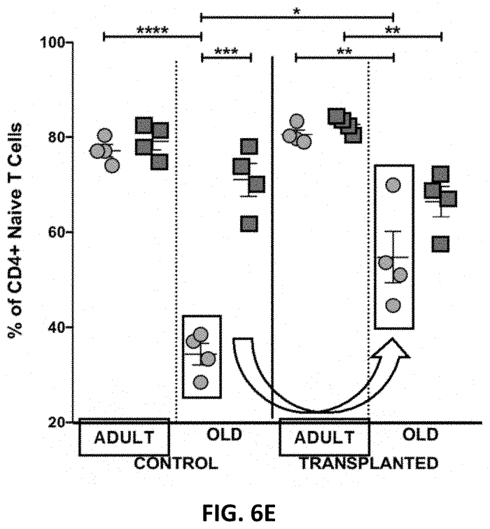

Old females showed a highly statistically significant decrease in CD4+ naïve T cells with respect to adult females (P<0.0001) (FIGS. 6A and 6E). The decrease in CD4+ naïve T cells in these mice resulted in a parallel and significant increases in activated CD4+ T cell effectors (P=0.0062), CD4+ effector/memory (P<0.0001) as well as CD4+ central memory T cells (P=0.0297) (FIGS. 6A-6D). Cells were first gated for singlets, followed by live cells, CD45+ cells, CD3+ cells, CD4+ , and finally, for CD44 and CD62L expression (FIGS. 5A-5H). Each dot represents a mouse. Circular dots indicate females; square dots indicate males. * indicates that the P-value is less than 0.05. ** indicates that the P-value is less than <0.01. *** indicates that the P is less than 0.001. **** indicates that the P-value is less than 0.0001. Interestingly, old males did not show any significant change in these populations with respect to adult males (FIGS. 6B-6E).

Importantly, CD4+ naïve T cells in old females were significantly increased after thymus transplantation (P=0.0127) with respect to the untransplanted counterpart, while the population of CD4+ effector/memory T cells was significantly decreased (P=0.0321) (FIGS. 6A-6E). A significant decrease in the population of CD4+ effector/memory T cells was also observed in old males after thymus transplantation (P=0.0112) with respect to their untransplanted counterpart (FIGS. 6B-6E). No changes in CD4+ subpopulations were observed in both adult females and males after thymus transplantation (FIGS. 6B-6E). These preliminary results indicate that (1) old females have lower naïve T cell percentages than age-matched males in this model; (2) aged females have a more profound decline in naïve T cells than age-matched males in this model; and (3) transplantation of a low dose of thymus increase CD4+ naïve T cell percentages in old females while reducing the proportion of CD4+ effector/memory T cells.

### 7.3 Example 3: Human Autologous Thymic Tissue Transplant

A human neonatal or infant subject presents with an indication that requires a thymectomy, for example, a congenital heart defect that requires surgical correction and a thymectomy as part of the surgery. Thymic tissue is collected during the surgery, and is cut or minced into small fragments using aseptic technique. Optionally, the thymic tissue is cultured ex vivo and/or cryopreserved as disclosed herein. The thymic tissue fragments are suspended in a solution that contains a suitable pharmaceutically-acceptable excipient, diluent, or carrier, and is injected into one or more lymph nodes of the subject as disclosed herein (e.g., using a minimally-invasive under the guidance of ultrasound). One month or several months later, a sample of the subject's peripheral blood is evaluated to determine whether the level of peripheral blood T cells and/or naïve T cells is higher compared to a comparable subject that did not receive the injection, or higher compared to the level in the subject before the fragments were injected. If the level of peripheral blood T cells and/or naïve T cells is still considered too low, more thymic tissue can be transplanted until an adequate number of peripheral blood T cells and/or naïve T cells is achieved.

### 7.4 Example 4: Thymic Tissue Transplant into a Human Subject

A human subject presents with a condition that affects function of the thymus, for example, age-related thymic atrophy. A suitable donor is identified, for example, cadaveric donor with an intact thymus that is HLA-matched to the subject. Thymic tissue is harvested from the donor and is cut or minced into small fragments using aseptic technique. Optionally, the thymic tissue is cultured ex vivo and/or cryopreserved as disclosed herein. The thymic tissue fragments are suspended in a solution that contains a suitable pharmaceutically-acceptable excipient, diluent, or carrier, and is injected into one or more lymph nodes of the subject as disclosed herein (e.g., using a minimally-invasive under the guidance of ultrasound). The subject is optionally administered an immunosuppressive regimen to improve graft acceptance. One month or several months later, a sample of the subject's peripheral blood is evaluated to determine whether the level of peripheral blood T cells and/or naïve T cells is higher compared to a comparable subject that did not receive the injection, or higher compared to the level in the subject before the fragments were injected. If the level of peripheral blood T cells and/or naïve T cells is still considered too low, more thymic tissue can be transplanted until an adequate number of peripheral blood T cells and/or naïve T cells is achieved.

Although the presently disclosed subject matter and its advantages have been described in detail, it should be understood that various changes, substitutions, and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which is incorporated herein by reference in their entireties for all purposes.

What is claimed is:

1. A method for preserving or restoring a thymic function of a subject in need thereof, the method comprising delivering thymic tissue into at least one lymph node of the subject, wherein the thymic tissue is autologous to the subject.

2. The method of claim 1, wherein the subject has a congenital heart defect.

3. The method of claim 1, wherein the subject has received or is receiving an open heart surgery.

4. The method of claim 1, wherein a thymectomy surgery was performed on the subject when the subject was a neonate or an infant.

5. The method of claim 1, wherein the subject is a neonate, an infant, a child, an adolescent, or an adult.

6. The method of claim 1, wherein the subject has received or is receiving a thymectomy surgery.

7. The method of claim 6, wherein the thymic tissue is obtained from the subject during the thymectomy surgery or before the thymectomy surgery.

8. The method of claim 1, wherein the thymic tissue comprises minced thymic fragments.

9. The method of claim 1, wherein the thymic tissue is cultured ex vivo before the delivering.

10. The method of claim 9, wherein the thymic tissue is cultured ex vivo for at least 24 hours before the delivering.

11. The method of claim 1, wherein the delivering comprises delivering the thymic tissue into the at least one lymph node of the subject through a needle.

12. The method of claim 6, wherein the delivering comprises delivering the thymic tissue into the at least one lymph node during or after the thymectomy surgery.

13. The method of claim 12, wherein the delivering comprises delivering the thymic tissue into the at least one lymph node after the thymectomy surgery, and wherein the thymic tissue is cryopreserved before the delivering the thymic tissue into the at least one lymph node.

14. The method of claim 13, wherein the delivering comprises delivering the cryopreserved thymic tissue into the at least one lymph node about 1 week or more after the thymectomy surgery.

15. The method of claim 1, wherein:
(a) the thymic tissue comprises an amount of thymic tissue effective to restore the thymic function of the subject; or
(b) the thymic tissue comprises an amount of thymic tissue effective to expand in the at least one lymph node to generate expanded thymic tissue, wherein the expanded thymic tissue restores the thymic function of the subject.

16. The method of claim 1, wherein the thymic tissue (i) comprises at least about 0.1 gram or up to about 20 grams; and/or (ii) comprises a size of at least about 0.1 cm³ or up to about 20 cm³.

17. The method of claim 1, wherein the at least one lymph node comprises at least two lymph nodes.

18. The method of claim 1, wherein the thymic tissue comprises at least about a third of full thymus weight of the subject.

19. A kit for preserving or restoring a thymic function of a subject, comprising thymic tissue, and a tool for delivery of the thymic tissue to at least one lymph node of the subject, wherein the thymic tissue is autologous to the subject.

20. A method comprising delivering a thymic tissue into a lymph node of a subject, wherein a frequency of CD45+ peripheral blood cells in the subject that are T cells is increased by at least about 5% over a frequency of CD45+ peripheral blood cells in the subject that are T cells before the delivering.

21. The method of claim 20, wherein a frequency of CD3+ peripheral blood cells in the subject that are naïve T cells is increased by at least about 5% over a frequency of CD3+ peripheral blood cells in the subject that are naïve T cells before the delivering.

22. The method of claim 20, wherein:
(a) the frequency of CD45+ peripheral blood cells in the subject that are T cells is at least about 20% after the delivering;
(b) a concentration of T cells in peripheral blood of the subject is increased by at least about 5% relative to a concentration of T cells in peripheral blood of the subject before the delivering;
(c) a frequency of CD3+ peripheral blood cells in the subject that are naïve T cells is at least about 20% after the delivering; or
(d) a concentration of naïve T cells in peripheral blood of the subject is increased by at least about 5% relative to a concentration of naïve T cells in peripheral blood of the subject before the delivering.

23. The method of claim 20, wherein the thymic tissue comprises at least about 7 grams of thymic tissue.

24. The method of claim 20, wherein the subject is at least about 40 years of age.

25. The method of claim 20, wherein the thymic tissue comprises at least about 50 milligrams of thymic tissue per kilogram of body weight of the subject.

26. The method of claim 20, wherein the subject has a condition that affects the thymus.

27. The method of claim 26, wherein the condition that affects the thymus comprises myasthenia gravis, pure red cell aplasia, hypogammaglobulinemia, thymus cancer, thymoma, type A thymoma, type B thymoma, an autoimmune disease, T cell-mediated autoimmunity, T cell lymphopenia, thymic atrophy, age-related thymic atrophy, thymic cyst, thymic hyperplasia, thymic hypoplasia, thymic aplasia, thymic dysplasia, severe combined immunodeficiency, Nezelof syndrome, Wiscott-Aldrich syndrome, DiGeorge syndrome, recurrent infection, recurrent viral infection, premature immunologic aging, or cancer.
